(19) United States
(12) Patent Application Publication
(10) Pub. No.: US 2013/0027666 A1
(43) Pub. Date: Jan. 31, 2013

(54) OPHTHALMIC APPARATUS

(71) Applicants: Satoshi Aikawa, Yokohama-shi (JP); Hideyuki Ohban, Kawaguchi-shi (JP); Yohei Saito, Chigasaki-shi (JP); Yoshitaka Nakano, Kawasaki-shi (JP)

(72) Inventors: Satoshi Aikawa, Yokohama-shi (JP); Hideyuki Ohban, Kawaguchi-shi (JP); Yohei Saito, Chigasaki-shi (JP); Yoshitaka Nakano, Kawasaki-shi (JP)

(73) Assignee: CANON KABUSHIKI KAISHA, Tokyo (JP)

(21) Appl. No.: 13/559,104

(22) Filed: Jul. 26, 2012

(30) Foreign Application Priority Data
Jul. 29, 2011 (JP) 2011-167053
Jul. 29, 2011 (JP) 2011-167054
Jul. 29, 2011 (JP) 2011-167055

(51) Int. Cl. A61B 3/10

(52) U.S. Cl. 351/214; 351/221

(57) ABSTRACT

An ophthalmic apparatus includes an optical system configured to illuminate a subject's eye with light generated by a light source, a first reflection portion including a reflection surface for reflecting the light generated by the light source and a transmission portion, and a light amount detection unit configured to detect an amount of light generated by the light source via the transmission portion. The first reflection portion is disposed in a direction opposite to a direction of the light generated by the light source toward the subject's eye.

### BACKGROUND OF THE INVENTION

1. Field of the Invention

The present invention relates to an ophthalmic apparatus.

2. Description of the Related Art

Japanese Patent Application Laid-Open No. 2003-70746 discuses, as a light source unit of an illumination optical system, a configuration in which a condenser lens, a flash illumination light source, a condenser lens, a continuous illumination light source, and a reflector are sequentially arranged from a fundus side. Japanese Patent Application Laid-Open No. 2003-70746 further discuses a configuration in which, in light emitted from the continuous illumination light source, a light flux output to a side opposite to the fundus is reflected by the reflector to travel toward the fundus. The reflector is formed into a concave shape, and configured to condense and project the light flux output from the continuous illumination light source to the side opposite to the fundus.

During photographing of the fundus, brightness varies from one subject to another, and there is an individual difference between light sources. Such differences cause variance in the brightness of a captured image. To eliminate the variance, the brightness of the fundus is detected in advance, and an amount of emitted light needs to be adjusted according to the brightness. The adjustment of the amount of emitted light requires detection of the amount of emitted light. The light source is optimized to illuminate a subject's eye, and light near an optical axis of the light source is accordingly more stable than light not near the optical axis. It is therefore desirable to dispose a detection unit for detecting the amount of emitted light on an illumination optical axis to improve detection accuracy.

However, according to the configuration discussed in Japanese Patent Application Laid-Open No. 2003-70746, the fundus side of the flash illumination light source is set as an illumination optical path, and the continuous illumination light source and the reflector are arranged on the side opposite to the fundus. Thus, it has been difficult to dispose the detection unit on the optical axis of the flash illumination light source. As a result, the configuration discussed in Japanese Patent Application Laid-Open No. 2003-70746 has been an obstacle to the improvement of the detection accuracy.

### SUMMARY OF THE INVENTION

The present invention is directed to a technique for accurate detecting an amount of light emitted from a light source. The present invention is further directed to provision of operation effects derived from exemplary embodiments described below, which are not acquired from the conventional technology.

According to an aspect of the present invention, an ophthalmic apparatus includes an optical system configured to illuminate a subject's eye with light generated by a light source, a first reflection portion including a reflection surface for reflecting the light generated by the light source and a transmission portion, and a light amount detection unit configured to detect an amount of light generated by the light source via the transmission portion, wherein the first reflection portion is disposed in a direction opposite to a direction of the light generated by the light source toward the subject's eye.

According to the present invention, the light amount detection accuracy can be improved.

Further features and aspects of the present invention will become apparent from the following detailed description of exemplary embodiments with reference to the attached drawings.

### BRIEF DESCRIPTION OF THE DRAWINGS

The accompanying drawings, which are incorporated in and constitute a part of the specification, illustrate exemplary embodiments, features, and aspects of the invention and, together with the description, serve to explain the principles of the invention.

FIG. 1 schematically illustrates an example of a configuration of an ophthalmic apparatus according to an exemplary embodiment of the present invention.

FIG. 2 schematically illustrates an example of an electric connection relationship of the ophthalmic apparatus.

FIG. 3 schematically illustrates an example of a power source included in the ophthalmic apparatus.

FIG. 4 (including FIGS. 4A and 4B) is a flowchart illustrating an example of an operation of the ophthalmic apparatus.

FIG. 5 schematically illustrates an example of a mirror included in the ophthalmic apparatus.

FIG. 6 schematically illustrates an example of a configuration of the ophthalmic apparatus.

FIG. 7 schematically illustrates an example of a ring slit included in the ophthalmic apparatus.

FIG. 8 schematically illustrates an example of a configuration of a light source unit of the ophthalmic apparatus.

FIG. 9 is a schematic sectional view illustrating an example of the configuration of the light source unit of the ophthalmic apparatus.

FIG. 10 schematically illustrates a behavior of light in the example of the configuration of the light source unit of the ophthalmic apparatus.

FIG. 11 is a schematic sectional view illustrating an example of the configuration of the light source unit of the ophthalmic apparatus.

FIG. 12 schematically illustrates a behavior of light in the example of the configuration of the light source unit of the ophthalmic apparatus.

FIGS. 13A and 13B schematically illustrate examples of configurations of openings of the ophthalmic apparatus.

FIG. 14 is a schematic sectional view illustrating an example of the configuration of the light source unit of the ophthalmic apparatus.

FIG. 15 schematically illustrates a behavior of light in the example of the configuration of the light source unit of the ophthalmic apparatus.

FIG. 16 is a schematic sectional view illustrating an example of the configuration of the light source unit of the ophthalmic apparatus.

FIG. 17 schematically illustrates a behavior of light in the example of the configuration of the light source unit of the ophthalmic apparatus.

FIG. 18 schematically illustrates an example of a configuration of the ophthalmic apparatus.

FIG. 19 schematically illustrates an example of a configuration of the light source unit of the ophthalmic apparatus.

FIG. 20 schematically illustrates a behavior of light in the example of the configuration of the light source unit of the ophthalmic apparatus.

FIG. 21 schematically illustrates an example of a configuration of the light source unit of the ophthalmic apparatus.

FIG. 22 schematically illustrates a behavior of light in the example of the configuration of the light source unit of the ophthalmic apparatus.

FIG. 23 schematically illustrates an example of a configuration of the ophthalmic apparatus.

FIG. 24 schematically illustrates an example of a configuration of the light source unit of the ophthalmic apparatus.

FIG. 25 schematically illustrates a behavior of light in the example of the configuration of the light source unit of the ophthalmic apparatus.

FIG. 26 schematically illustrates an example of a configuration of the ophthalmic apparatus.

FIG. 27 schematically illustrates an example of a detailed configuration of a photographic light source unit and an observation light source unit included in the ophthalmic apparatus.

FIG. 28 schematically illustrates a behavior of light in the example of the configuration of the light source unit of the ophthalmic apparatus.

FIG. 29 schematically illustrates an example of a configuration of the ophthalmic apparatus.

FIG. 30 schematically illustrates an example of a detailed configuration of a photographic light source unit and an observation light source unit included in the ophthalmic apparatus.

FIG. 31 schematically illustrates a behavior of light in the example of the configuration of the light source unit of the ophthalmic apparatus.

### DESCRIPTION OF THE EMBODIMENTS

Various exemplary embodiments, features, and aspects of the invention will be described in detail below with reference to the drawings.

A fundus camera according to a first exemplary embodiment of the present invention will be described in detail with reference to the accompanying drawings.

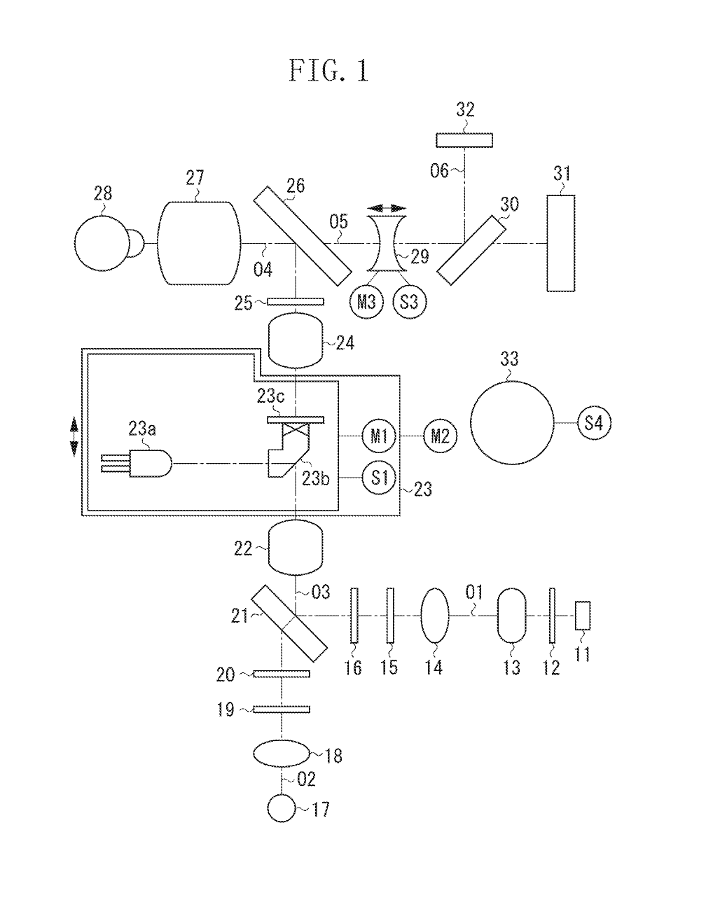

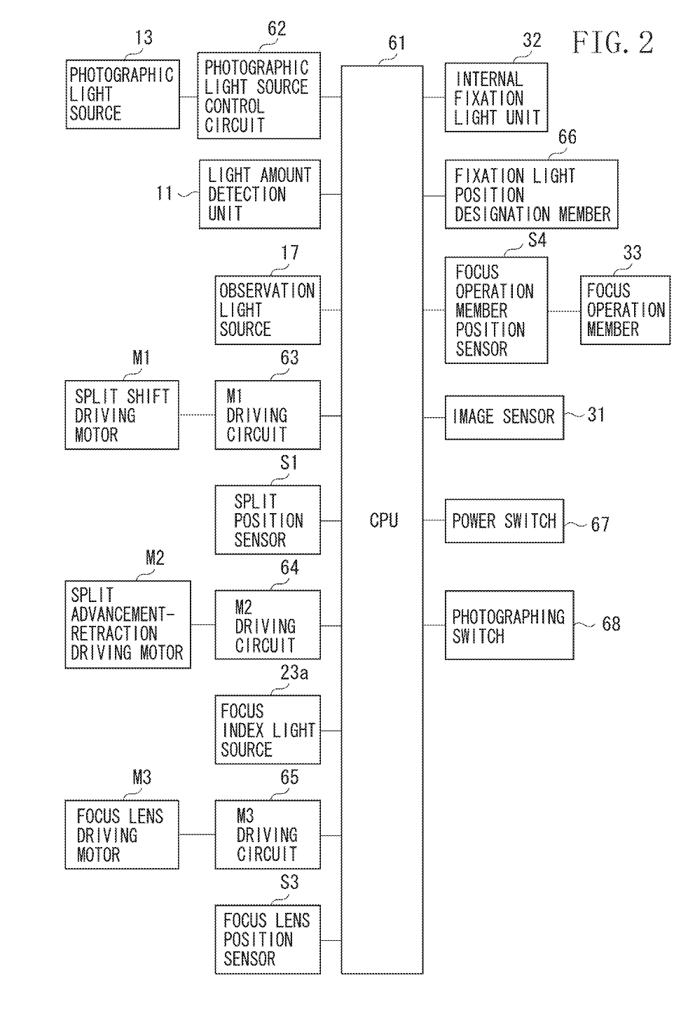

FIG. 1 schematically illustrates a configuration of an ophthalmic apparatus. FIG. 2 schematically illustrates an example of an electric connection relationship of the ophthalmic apparatus. A fundus camera (i.e., the ophthalmic apparatus) illustrated in FIG. 1 roughly includes a photographic light source unit O1, an observation light source unit O2, an illumination optical system O3, a photographic/illumination optical system O4, a photographic optical system O5, and an internal fixation light section O6.

A light flux emitted from the photographic light source unit O1 or the observation light source unit O2 passes through the illumination optical system O3 and the photographic/illumination optical system O4 to illuminate a fundus of a subject's eye. An image of the fundus is formed on an image sensor through the photographic/illumination optical system O4 and the photographic optical system O5. In other words, the photographic light source unit O1, the illumination optical system O3, and the photographic/illumination optical system O4 constitute an example of an optical system for illuminating the subject's eye with light generated by the light source. In addition, the observation light source unit O2, the photographic/illumination optical system O4, and the photographic optical system O5 constitute an example of an optical system for illuminating the subject's eye with the light generated by the light source.

The photographic light source unit O1 includes components described below to generate ring illumination of white light. Alight amount detection unit 11 is a sensor using known photoelectric conversion of a silicon photodiode (SPD) or a photodiode (PD). The light amount detection unit 11 includes an integration circuit that calculates an amount of light by integrating an output of the sensor such as a PD.

A mirror 12 is configured by depositing aluminum or silver on a glass plate or using an aluminum plate. For example, the mirror 12 is formed planar within a processing limit range to realize uniform photographic light sources. In other words, the mirror 12 is a planar mirror. In this case, the planar form is a concept including processing errors. Therefore, it does not mean only a completely planar form. The mirror 12 is disposed, for example, in a direction opposite to a direction from a photographic light source 13 toward a subject's eye 28. The mirror 12 will be described in detail below referring to FIG. 5.

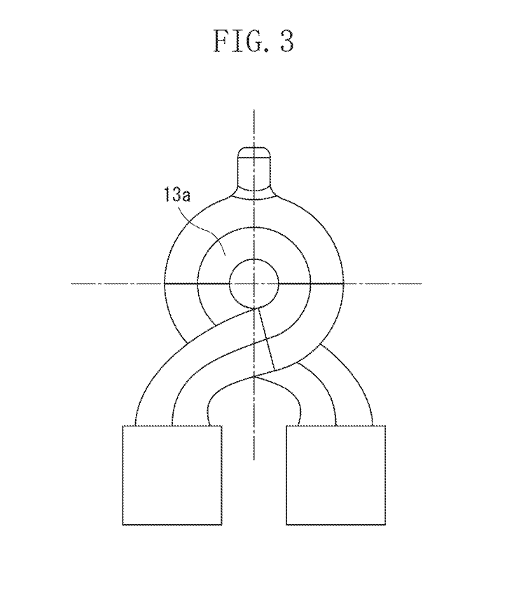

The photographic light source 13 emits light by applying a voltage to a glass tube enclosing xenon therein, and enables acquisition of white light of intensity sufficient to record a fundus image during photographing. Recently, an increase in the light amount of a light emitting diode (LED) has been conspicuous, and even an annularly arranged LED array can realize the photographic light source 13. FIG. 3 schematically illustrates an example of the photographic light source 13. As illustrated in FIG. 3, a light emitting portion of the photographic light source 13 is annular, and the photographic light source 13 includes an annular light emitting portion 13a that radially emits light.

A photographic condenser lens 14 is a general spherical lens. A photographic ring slit 15 is a flat plate including an annular opening. For example, the ring slit 15 is disposed in a position roughly conjugate with an anterior segment of the subject's eye 28. A photographic crystalline lens baffle 16 is also a flat plate including an annular opening.

Light fluxes emitted from the photographic light source 13, that is, for example, the xenon tube (Xe tube), include a light flux toward the fundus and a light flux, which is emitted to a side opposite to the direction toward the fundus and reflected by the mirror 12 to travel toward the fundus. In other words, the light flux emitted from the photographic light source 13 and the light flux reflected by the mirror 12 enter the subject's eye 28, and accordingly the amount of light entering the subject's eye 28 is larger than when there is no mirror 12.

Thus, the amount of light emitted from the photographic light source 13 can be smaller than when there is no mirror 12. The mirror 12 is formed planar, preventing light unevenness, and there is no restriction on a distance to the photographic light source 13.

The light flux emitted from the photographic light source 13 and the light flux reflected by the mirror 12 toward the subject's eye are condensed toward the fundus by the photographic condenser lens 14, and formed to be annular by the ring slit 15 during passage through the anterior eye segment. In addition, the photographic crystalline lens baffle 16 limits a light flux projected to a crystalline lens of the subject's eye 28, thereby preventing unnecessary projection of reflected light from the crystalline lens of the subject's eye 28 in the fundus image.

The observation light source unit O2 includes components described below to generate ring illumination of infrared light. An observation light source 17, which is a light source such as a halogen lamp or an LED capable of continuously emitting light, emits infrared light based on element characteristics or via a filter (not illustrated). An observation condenser lens 18 is a general spherical lens. An observation ring slit 19 is a flat plate including an annular opening. An observation crystalline lens baffle 20 is also a flat plate including an annular opening.

The observation light source unit O2 is different from the photographic light source unit O1 only in the type of the light source. The light flux is condensed by the observation condenser lens 18, and a shape of the light flux is rectified at the anterior eye segment by the observation ring slit 19. Accordingly, unnecessary projection of reflected light from the crystalline lens in the fundus image is prevented by the observation crystalline lens baffle 20.

The illumination optical system O3 includes components described below to relay the light fluxes generated by the photographic light source unit O1 and the observation light source unit O2, and generates an index image for focusing the fundus image. A dichroic mirror 21 transmits infrared light while reflecting visible light. The light flux of the visible light generated by the photographic light source unit O1 is reflected by the dichroic mirror 21, while the light flux of the infrared light generated by the observation light source unit O2 is transmitted through the dichroic mirror 21 to be guided to the illumination optical system O3. Illumination relay lenses 22 and 24 form an image of the ring illumination on the subject's eye 28.

A split unit 23 includes a focus index light source 23a for projecting a focus index, a prism 23b for dividing the light source, and a focus index mask 23c indicating an outer shape of the focus index. The split unit 23 further includes a moving mechanism for shifting and moving the focus index in an optical axis direction by entering the focus index light source 23a, the prism 23b, and the focus index mask 23c into the illumination optical system O3 and moving them to a direction indicated by an arrow in the FIG. 1 (i.e., the optical axis direction) during observation. The split unit 23 further includes an advancement and retraction mechanism for retracting from the illumination optical system O3 during photographing.

A split shift driving motor M1 shifts and drives the split unit 23 in the arrow direction in FIG. 1 to set the focus index in focus. A split position sensor S1 detects a stop position of the split unit 23.

A split advancement-retraction driving motor M2 advances or retracts the split unit 23 into or from the illumination optical system O3. The split advancement-retraction driving motor M2 advances the split unit 23 into the illumination optical system O3 during fundus observation to project a split index in an observation image. On the other hand, during photographing, the split advancement-retraction driving motor M2 retracts the split unit 23 from the illumination optical system O3 to perform control to prevent projection of the focus index in a captured image. A cornea baffle 25 prevents unnecessary projection in the fundus image of reflected light from a cornea of the subject's eye 28.

The photographic/illumination optical system O4 includes components described blow to project an illumination light flux to the fundus of the subject's eye 28 and acquire a fundus image of the subject's eye 28. A perforated mirror 26 has its outer circumference set as a mirror and its center set as a hole. The light flux guided from the illumination optical system O3 is reflected by the mirror portion of the perforated mirror 26 to illuminate the fundus of the subject's eye 28 via an objective lens 27. The reflected light from the fundus returns through the objective lens 27, and then passes through the center hole of the perforated mirror 26 to be guided to the photographic optical system O5.

The photographic optical system O5 includes the components described below to adjust the focus of the fundus image of the subject's eye and form the fundus image of the subject's eye on the image sensor. A focus lens 29 adjusts the focus of the photographic light flux passed through the center hole of the perforated mirror 26. The focus lens 29 moves in the arrow direction in FIG. 1 (i.e., the optical axis direction) to adjust the focus. A focus lens driving motor M3 drives the focus lens 29 to set it in focus, and a focus lens position sensor S3 detects a stop position of the focus lens 29. An image sensor 31 photoelectrically converts photographic light. An electric signal acquired by the image sensor is analog-to-digital (A/D) converted into digital data by a processing circuit (not illustrated). For example, the digital data is displayed on a display (not illustrated) during infrared light observation, and recorded in a recording medium (not illustrated) after photographing.

The internal fixation light section O6 includes an internal fixation light unit 32 located facing an optical path split from the photographic optical system O4 by a half mirror 30. The internal fixation light unit 32 includes, for example, a plurality of LEDs, and turns on an LED at a position corresponding to a visual fixation unit selected by an examiner using a fixation light position designation member 66 described below. When a subject fixes its view on the lit LED, the examiner can acquire a fundus image of a desirable direction.

A focus operation member 33 can be operated by the examiner. When the examiner operates the focus detection member 33, a focus operation member position sensor S4 can detect a stop position of the focus operation member 33.

FIG. 2 schematically illustrates an example of an electric connection relationship of the ophthalmic apparatus. A central processing unit (CPU) 61 controls entire operations described below of the fundus camera. Other processing units can be used in place of the CPU 61. For example, in place of the CPU, a processing unit such as a field programmable gate array (FPGA) can be used. A photographic light source control unit 62 charges a capacitor (not illustrated) with energy to cause the photographic light source 13 to emit light before the photographing. The light amount detection unit 11 detects the amount of light emitted from the photographic light source 13, and instructs the CPU 61 to stop light emission, for example, when the amount of light emitted from the photographic light source 13 reaches the amount of emitted light limited by the CPU 61, and the light emission of the photographic light source 13 is stopped via a photographic light source control circuit 62. The photographic light source control circuit 62 causes the photographic light source 13 to emit light by discharging the electric energy charged during photographing.

An M1 driving circuit 63 drives the split shift driving motor M1 so that the split unit 23 can move to a position corresponding to an output of the focus operation member position sensor S4. An M2 driving circuit 64 drives the split advancement-retraction driving motor M2 so that the split unit 23 can advance into or retract from the illumination optical system O3 before and after the photographing. An M3 driving circuit 65 drives, as in the case of the M2 driving circuit 64, the focus lens driving motor M3 so that the focus lens 29 can move to the position corresponding to the output of the focus operation member position sensor S4. A power switch 67 is used for selecting a power state of the fundus camera, and a photographing switch 68 is used for executing photographing by the fundus camera.

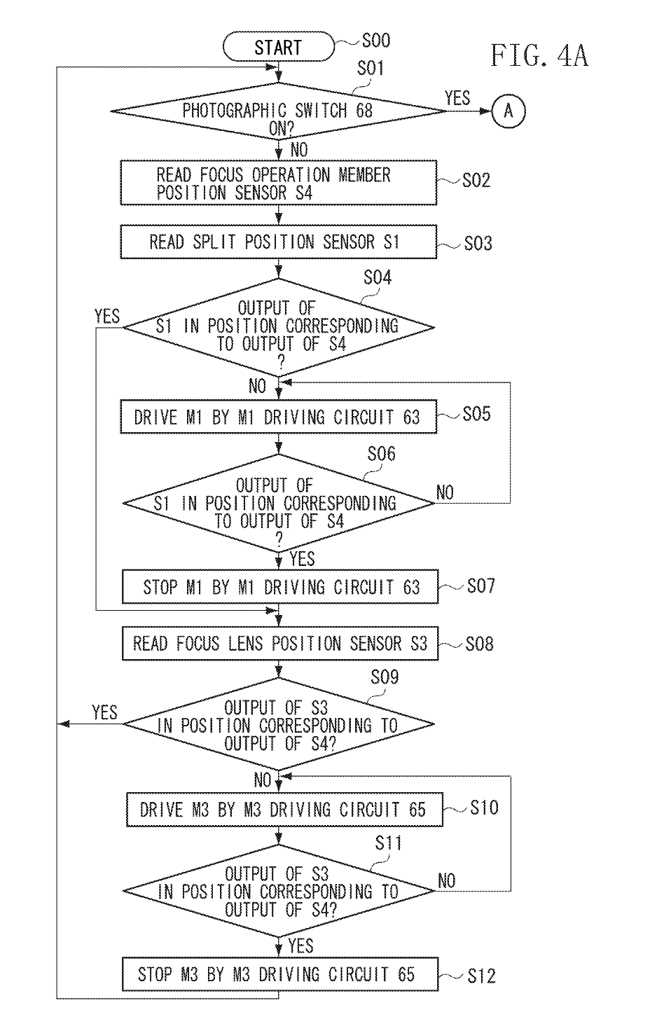

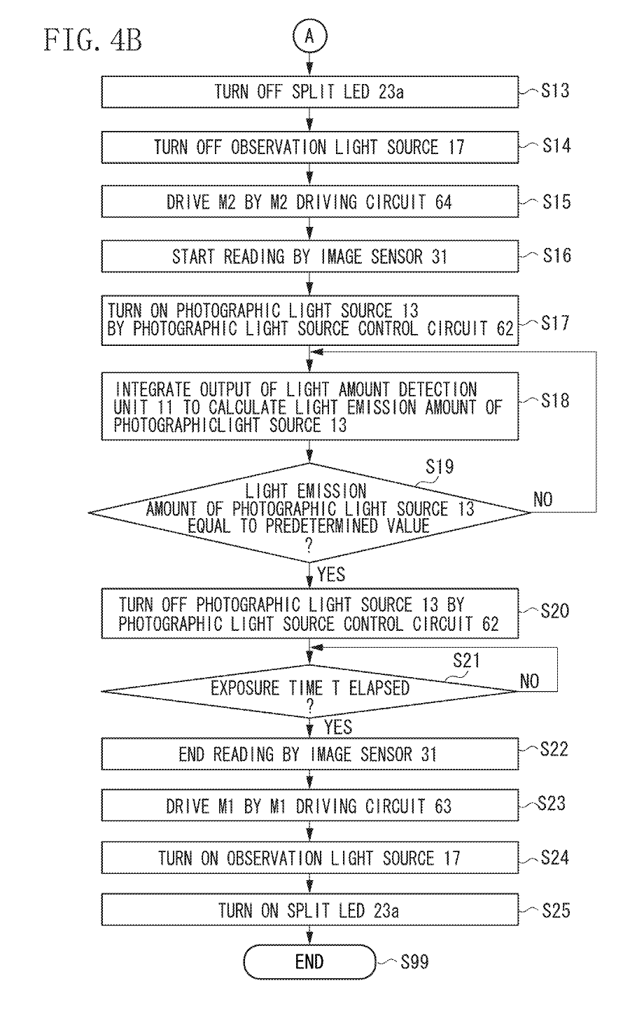

FIG. 4 (including Fogs. 4A and 4B) is a flowchart illustrating an example of an operation of the ophthalmic apparatus according to the present exemplary embodiment. Specifically, FIG. 4 is a flowchart mainly illustrating an example of an operation relating to focus adjustment.

In step S00, the processing sequence is started when the power switch 67 turns power ON. In step S01, the CPU 61 checks whether the photographing switch 68 is ON. If the photographing switch 68 is ON (YES in step S01), the processing proceeds to step S13. Whereas if not (NO in step S01), the processing proceeds to step S02.

In step S02, the CPU 61 reads an output of the focus operation member position sensor S4.

In step S03, the CPU 61 reads an output of the split position sensor S1. The execution order of steps S02 and S03 can be reversed. Then, in step S04, the CPU 61 checks whether the output of the split position sensor S1 is in a position corresponding to the output of the focus operation member position sensor S4. If the output is in the corresponding position (YES in step S04), the processing proceeds to step S08. Whereas if not (NO in step S04), the processing proceeds to step S05.

In step S05, the M1 driving circuit 63 drives the split shift driving motor M1 so that the split unit 23 can move to the position corresponding to the focus operation member position sensor S4.

In step S06, the CPU 61 reads an output of the split position sensor S1 to check whether the split unit 23 moved in step S05 has moved to the position corresponding to the focus operation member position sensor S4. If the split unit 23 has not moved to the corresponding position (NO in step S06), the processing returns to step S05. If the split unit 23 has moved to the corresponding position (YES in step S06), the processing proceeds to step S07.

In step S07, the M1 driving circuit 63 stops the split shift driving motor M1, and then the processing proceeds to step S08.

In step S08, the CPU 61 reads an output of the focus lens position sensor S3.

In step S09, the CPU 61 checks whether the output of the focus lens position sensor S3 is in a position corresponding to the output of the focus operation member position sensor S4. If the output is in the corresponding position (YES in step S09), the processing returns to step S01. Whereas if not (NO in step S09), the processing proceeds to step S10.

In step S10, the M3 driving circuit 65 drives the focus lens driving motor M3 so that the focus lens 29 can move to the position corresponding to the focus operation member position sensor S4.

In step S11, the CPU 61 reads an output of the focus lens position sensor S3 to check whether the focus lens 29 moved in step S10 has moved to the position corresponding to the focus operation member position sensor S4. If the focus lens 29 has not moved to the corresponding position (NO in step S11), the processing returns to step S10. Whereas if the focus lens 29 has moved to the corresponding position (YES in step S11), the processing proceeds to step S12.

In step S12, the M3 driving circuit 63 stops the focus lens driving motor M3, and then the processing returns to step S01.

In step S13, since the photographic switch 68 is turned ON, first, the CPU 61 turns off the split LED 23a.

In step S14, the CPU 61 turns off the observation light source 17.

In step S15, the M2 driving circuit 64 drives the split advancement-retraction driving motor M2 to retract the split unit 23 from the illumination optical system O3.

In step S16, the image sensor 31 starts reading the fundus image.

In step S17, the photographic light source control circuit 62 turns on the photographic light source 13.

In step S18, the light amount detection unit 11 integrates an output from its own PD to calculate the amount of light emitted from the photographic light source 13.

In step S19, the CPU 61 checks whether the amount of light emitted from the photographic light source 13 has reached a predetermined value suited for fundus photographing. When the amount of light calculated in step S18 has not reached the predetermined value (NO in step S19), the CPU returns the processing to step S18 while causing the photographic light source 13 to continue light emission. On the other hand, when the amount of light calculated in step S18 has reached the predetermined value (YES in step S19), the processing proceeds to step S20.

In step S20, the photographic light source control circuit 62 turns off the photographic light source 13. For example, the photographic light source control circuit 62 stops supplying of current to the photographic light source 13 to stop the light emission of the photographic light source 13. In other words, the photographic light source control circuit 62 is an example of a light emission control unit that controls the light emission of the light source according to the amount of light detected by the light amount detection unit.

Then, in step S21, the CPU 61 checks whether exposure time T has reached predetermined exposure time. When the exposure time T has not reached the predetermined exposure time (NO in step S21), the CPU 61 repeats the processing in step S21. When the exposure time T has reached the predetermined exposure time (YES in step S21), the processing proceeds to step S22.

In step S22, the reading from the image sensor 31 is completed. Though not described in detail, the image sensor 31 outputs image information as an electric signal by photoelectric conversion, and the output electric signal is subjected to electric processing such as AD conversion and then stored as electronic data.

In step S23, the M1 driving circuit 63 drives the split shift driving motor M1 to return the split unit 23 into the illumination optical system O3. In step S24, the CPU 61 lights the observation light source 17.

In step S25, the CPU 61 lights the split LED 23a, the processing returns to the photographing preparation state, and the processing sequence in the flowchart in FIG. 4 is completed.

FIG. 5 schematically illustrates an example of the mirror 12 included in the ophthalmic apparatus.

FIG. 5 is a plan view of the mirror 12 seen from a reflection surface side when aluminum is deposited on glass. Reflection units 12a and 12b, on which aluminum is deposited, reflect a light flux emitted from the photographic light source 13 in a direction opposite to the subject's eye toward the subject's eye.

In the present exemplary embodiment, the aluminum is deposited on the glass to form the reflection portions. Not limited to the aluminum, however, any substance can be used to be deposited as long as the substance can reflect light.

A transmission portion 12c guides the light from the photographic light source 13 to the light amount detection unit 11. The transmission portion 12c transmits the light from the photographic light source 13. In other words, the mirror 12 is an example of a first reflection portion that includes a reflection surface to reflect light generated by the light source and a transmission portion. The reflection portions 12a and 12b are examples of the reflection surfaces. The first reflection portion is disposed in a direction opposite to the direction of the light generated by the light source toward the subject's eye. More specifically, the reflection surface is set in the direction of the light generated by the light source toward the subject's eye. The light amount detection unit 11 is an example of a light amount detection unit that detects the amount of light generated by the light source via the transmission portion. The light amount detection unit is disposed in the direction opposite to the direction of the light generated by the light source toward the subject's eye. In other words, the first reflection portion is disposed between the light amount detection unit and the light source.

For example, the light amount detection unit 11 is located on an optical axis (on optical axis of the photographic light source 13) of the optical system for illuminating the subject's eye 28 with the light generated by the light source in order to accurately detect the amount of light emitted from the photographic light source 13. In this case, the transmission portion 12c is also located on the optical axis of the photographic light source 13 to guide the light to the light amount detection unit 11.

To provide the transmission portion 12c, for example, in the center of the mirror 12, the center is masked to prevent aluminum from being deposited thereon during a deposition process of aluminum. A mask can be attached for each component. However, work for attaching the mask for each component is cumbersome, and may deteriorate position accuracy.

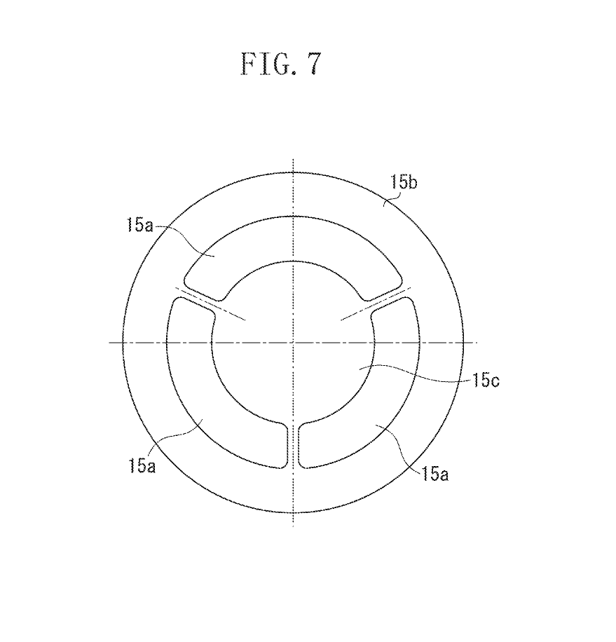

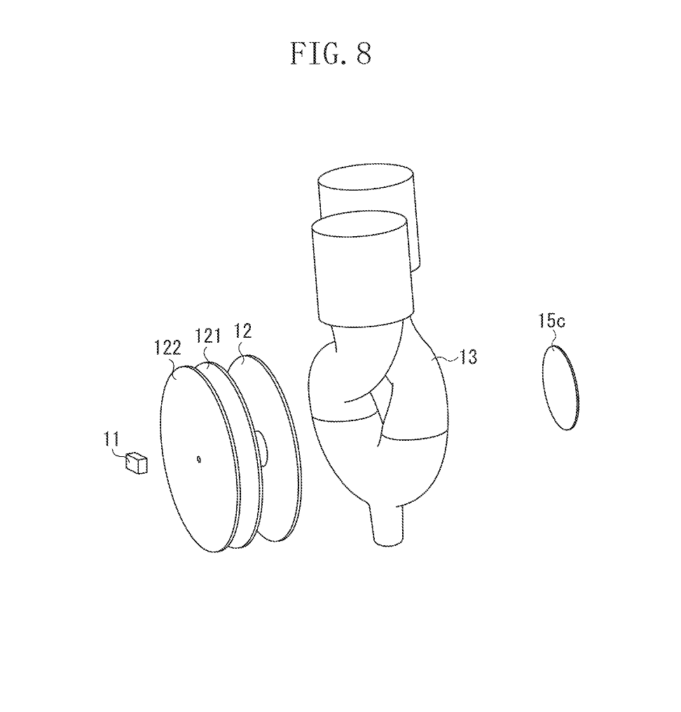

Thus, it is desirable to connect all masks to enable simultaneous attachment of the masks to many components. Accordingly, the masks have radial connection portions from the transmission portion 12c to the outer circumference, and transmission portions 12d and 12e for connection can be formed in the mirror 12. The transmission portions 12d and 12e are radially formed toward the outer circumference of the reflection portion (the mirror 12). These transmission portions, which are not necessary as optical components, are naturally formed to eliminate the cumbersome work. However, it has been experimentally confirmed that these units do not adversely affect the function of the mirror 12. In other words, FIG. 4 illustrates the transmission portions 12d and 12e arranged in the mirror 12. However, the mirror 12 can include only the transmission portion 12c without forming the transmission portions 12d and 12e. The reflection portions 12a and 12b included in the mirror 12 restrict entry of a light flux emitted from the photographic light source 13 directly travels to the light amount detection unit 11 (refer to FIG. 8 described below).

Shapes of the mirror 12 and the transmission portion 12c illustrated in FIG. 5 are circular. Not limited to the circular shape, however, various shapes can be employed.

According to the above-described present exemplary embodiment, the light amount detection unit 11 is disposed on the optical axis of the photographic light source 13. However, the configuration is not limited to this example, and the light amount detection unit 11 does not need to be completely located on the optical axis. For example, if the light amount detection unit 11 is located near the optical axis, the light amount detection unit 11 can receive stable light from near the optical axis to detect the amount of light. In this case, the transmission portion 12c is also located near the optical axis.

According to the present exemplary embodiment, the mirror 12 and the light amount detection unit 11 are arranged in the direction opposite to the direction from the photographic light source 13 toward the subject's eye 28. However, the arrangement is not limited to this configuration. For example, the mirror 12 and the light amount detection unit 11 can be arranged in the direction opposite to the direction from the observation light source 17 toward the subject's eye 28. In this case, the light amount detection unit 11 detects the amount of light emitted from the observation light source 17.

Further, according to the present exemplary embodiment, it is described that the light amount detection unit 11 is desirable to be disposed on the optical axis of the photographic light source 13. However, the arrangement is not limited to this configuration. For example, as long as the transmission portion 12c is located on the optical axis of the photographic light source 13 and a mirror for guiding the light transmitted through the transmission portion 12c to the light amount detection unit is included, the light amount detection unit 11 can be set in a position shifted from the optical axis. Such a configuration enables guiding of the stable light near the optical axis of the photographic light source 13 to the light amount detection unit 11.

According to the ophthalmic apparatus of the present exemplary embodiment, the mirror 12 including the transmission portion 12c is disposed in the direction opposite to the direction from the photographic light source 13 toward the subject's eye 28, and the light amount detection unit 11 detects the amount of light transmitted through the transmission portion 12c. Therefore, the light amount detection unit 11 can be disposed in a desired position. This configuration can realize accurate detection of the amount of light emitted from the light source. More specifically, by arranging the transmission portion 12c and the light amount detection unit 11 on the optical axis of the photographic light source 13, the amount of light emitted from the photographic light source 13 can be detected more accurately.

In addition to such effects, since the reflection portions 12a and 12b of the mirror 12 can direct the light, which is not directed to the subject's eye among the light beams emitted from the photographic light source 13, toward the subject's eye, the light generated by the photographic light source 13 can be effectively used.

Further, since the mirror 12 includes the reflection portions 12a and 12b and the transmission portion 12c, it contributes to the effect of accurately detecting the amount of emitted light using one single member and the effect of effectively using the light emitted from the photographic light source 13, enlargement of the apparatus can be prevented.

Further, rather than using one independent mask for each mirror 12 to form the transmission portion 12c, masks in which the one mask is connected and which can be simultaneously used for a plurality of mirrors 12 are used. Thus, cumbersome work and deterioration of mask position accuracy are prevented.

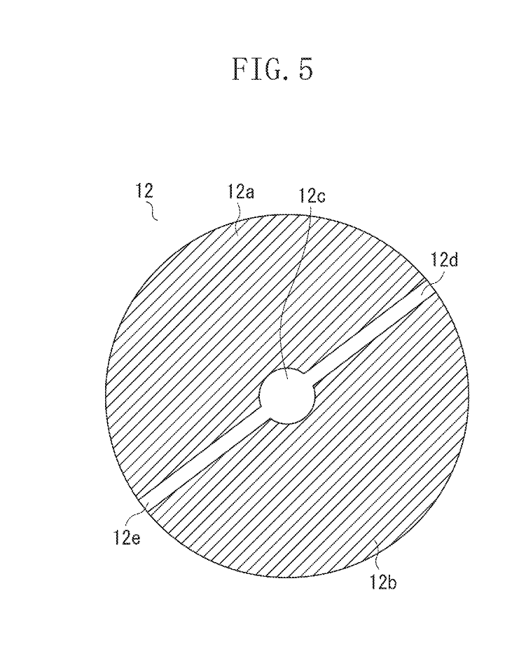

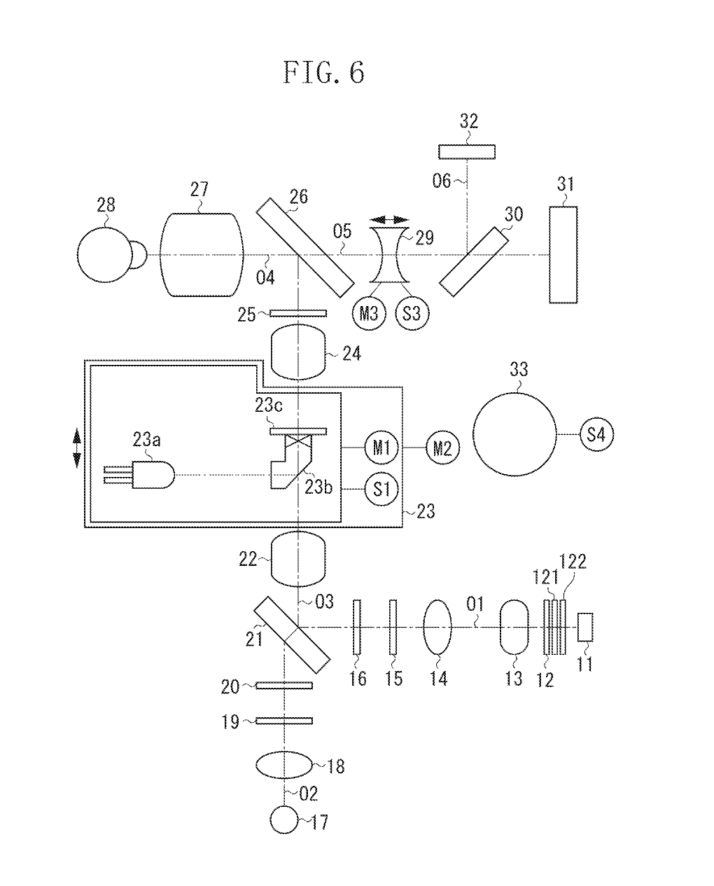

Next, referring to FIGS. 6 to 10, an ophthalmic apparatus according to a second exemplary embodiment will be described. In the first exemplary embodiment, the light emitted from the photographic light source 13 enters the light amount detection unit 11 via the transmission portion 12c. However, when intensity of the light emitted from the photographic light source 13 is strong, the light amount detection unit 11 cannot accurately detect the amount of light. One of the reasons is, for example, saturation of an output of an integration circuit included in the light mount detection unit.

Thus, the second exemplary embodiment is directed to the ophthalmic apparatus that can accurately detect the amount of light by causing reflected light to enter the light amount detection unit, which is different from the first exemplary embodiment, even when intensity of the light emitted from the photographic light source 13 is strong. More specifically, among light beams emitted from the photographic light source 13, light reflected on a predetermined reflection surface is received by the light amount detection unit 11. Since the light amount detection unit 11 receives the reflected light, the light amount detection unit 11 can detect the amount of emitted light more accurately than when directly receiving the strong light.

FIG. 6 schematically illustrates an example of a configuration of the ophthalmic apparatus according to the second exemplary embodiment. Components similar to those described in the first exemplary embodiment are denoted by the same reference numerals, and thus descriptions thereof will be omitted.

As can be understood from FIG. 6, the second exemplary embodiment is different from the first exemplary embodiment in that the ophthalmic apparatus includes alight flux restriction unit 121 and a light flux restriction unit 122. Among light fluxes transmitted through a transmission portion 12c in a mirror 12, the light flux restriction unit 121 restricts entry of, for example, direct light to the light amount detection unit 11. Among light beams transmitted through the light flux restriction unit 121, the light flux restriction unit 122 restricts traveling of, for example, light, which is reflected by an inner wall formed around the light amount detection unit 11 and causes diffused reflection light, to the light amount detection unit 11 side. The light flux restriction unit 121 and the light flux restriction unit 122 will be described in detail below referring to FIG. 9.

FIG. 7 schematically illustrates an example of a ring slit 15. The ring slit 15 includes an annular projection portion 15a for annularly projecting a light flux from the photographic light source, an annular light shielding portion 15b for restricting an outer size of the annular projection portion 15a, and a circular light shielding portion 15c for restricting an inner size of the annular projection portion. A center of the circular light shielding portion 15c is desirable to be located on the optical axis of the photographic light source 13. The circular light shielding portion 15c also functions as a circular reflection portion for reflecting light entered to itself. The ring slit 15 is made of, for example, stainless steel. The circular light shielding portion 15c only needs to be configured to reflect the light entered to itself. The material of the ring slit 15 is not limited to the stainless steel. As described above, the ring slit 15 is disposed in a position roughly conjugate with an anterior segment of a subject's eye 28, and includes a reflection portion configured to reflect a part of light emitted from the light source on an optical axis of an optical system.

Accordingly, a part of the light fluxes emitted from the photographic light source 13 to the ring slit 15 travels to a photographic crystalline lens baffle 16 via the annular projection portion 15a, while another part of the light fluxes emitted from the photographic light source 13 to the ring slit 15 is reflected by the circular light shielding portion 15c. The light reflected by the circular light shielding portion 15c is guided to the light amount detection unit 11. In other words, the circular light shielding portion 15c is an example of a second reflection portion for guiding the light generated by the light source to the light amount detection unit. As described above, the second reflection portion (the circular light shielding portion 15c) is disposed on the optical axis of the optical system and in a direction from the light source to the subject's eye 28. Thus, the ring sling 15 includes the second reflection portion on the optical axis of the optical system. In other words, the ring slit 15 is disposed on the position roughly conjugate with the anterior segment of the subject's eye 28, and includes the reflection portion configured to reflect apart of the light emitted from the light source on the optical axis of the optical system.

A reflectance and a surface shape of the circular light shielding portion 15c included in the ring slit 15 are controlled. For example, a reflectance is uniform or nearly uniform on a reflection surface of the circular light shielding portion 15c. In other words, the reflectance of the circular light shielding portion 15c is known.

FIGS. 8 and 9 illustrate an example of the light flux restriction unit included in the ophthalmic apparatus according to the present exemplary embodiment. FIG. 8 illustrates the light amount detection unit 11, the light flux restriction units 121 and 122, the mirror 12, the photographic light source 13, and the circular light shielding portion 15c included in the ring slit 15. For simplicity of the description, a condenser lens 14 is not illustrated. FIG. 9 illustrates a cross section of FIG. 8. As illustrated in FIG. 9, the mirror 12 includes a transmission portion 12c, and the light flux restriction units 121 and 122 respectively include openings 121a and 122a. The openings 121a and 122a are arranged to allow transmission of light reflected by the circular light shielding portion 15c. It is desirable for the openings 121a and 122a to be arranged on the optical axis of the photographic light source 13. The opening 121a is an example of a first opening through which the light can pass. The opening 122a is an example of a second opening through which the light can pass.

The light flux restriction unit 121 restricts entry of light directly entering to the light amount detection unit 11 from the photographic light source among light beams transmitted through the transmission portion 12c of the mirror 12. Accordingly, for example, the light flux restriction unit 121 is disposed such that a light shielding member 121b constituting the light flux restriction unit 121 can be located on a straight line connecting a light emitting portion of the photographic light source 13 with the light amount detection unit 11. In other words, the light flux restriction unit 121 is an example of a first restriction unit located on the straight line connecting the light emitting portion of the photographic light source with the light amount detection unit.

The light flux restriction unit 122 restricts traveling of light, which is reflected by the inner wall (indicated by an alternate long and short dash line illustrated in FIG. 9) having non-uniform reflection characteristics formed around the light amount detection unit 11 and causes diffused reflection light among light beams transmitted through the opening 121a of the light flux restriction unit 121, to the light amount detection unit 11 side. In other words, the light flux restriction unit 122 restricts entry of diffused reflection light to the light amount detection unit 11. Accordingly, for example, the light flux restriction unit 122 is disposed such that a light shielding member 122b constituting the light flux restriction unit 122 can be located on a straight line connecting the light emitting portion of the photographic light source 13 with the inner wall formed around the light amount detection unit 11. In other words, the light shielding member 122b is set in a position to block traveling of the light, which is transmitted through the transmission portion 12c and the opening 121a among the light beams emitted from the light emitting portion of the photographic light source 13, to the light amount detection unit 11 side. In other words, the light flux restriction unit 122 is an example of a second restriction unit that restricts entry of light other than light at least reflected by the second reflection portion among the light beams transmitted through the first opening to the light amount detection unit and includes, on the optical axis, a second opening through which the light can pass.

The light flux restriction units 121 and 122 also restrict entry of the diffused reflection light, which is emitted from the photographic light source 13 and reflected on the inner wall formed around the photographic light source 13, to the light amount detection unit 11.

As described above, arrangement of the light flux restriction units 121 and 122 can restrict the entry of the direct light from the photographic light source 13 to the light amount detection unit 11 and the entry of the diffused reflection light on the inner wall.

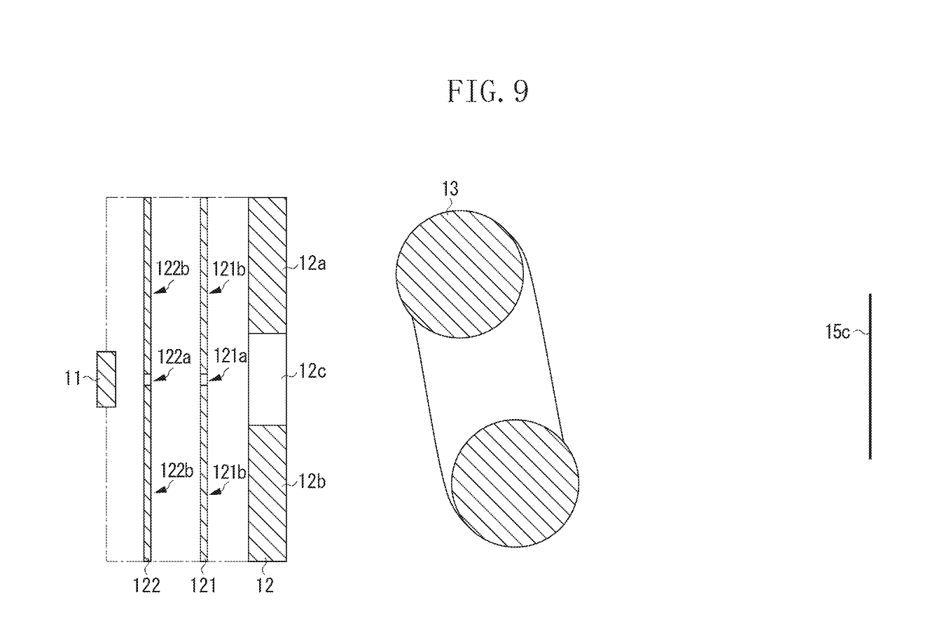

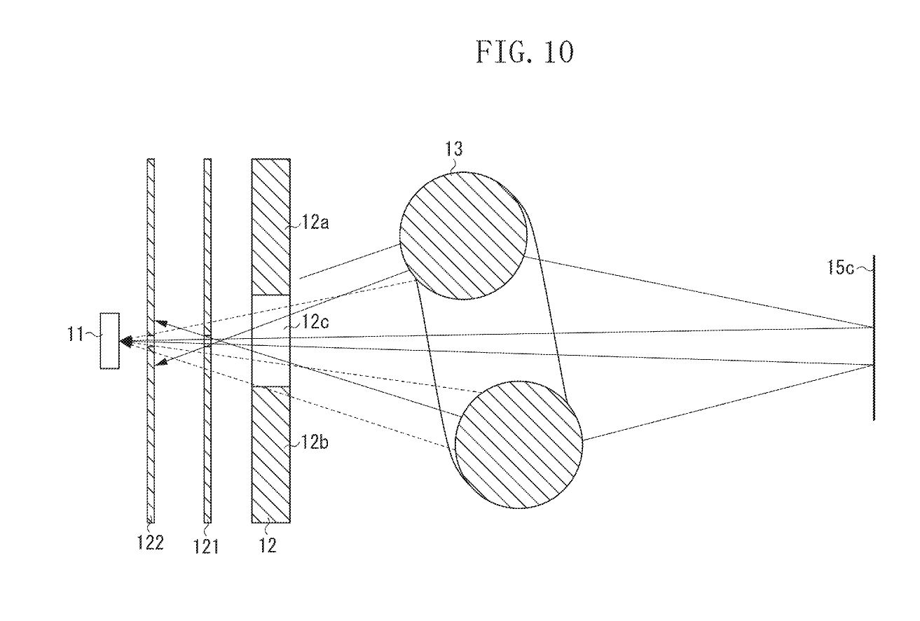

A specific behavior of the light emitted from the photographic light source 13 will be described below with reference to FIG. 10.

First, the photographic light source 13 emits light fluxes. Among the light fluxes emitted from the photographic light source 13, as illustrated in FIG. 10, a light flux reflected by the circular light shielding portion 15c passes through a gap of the photographic light source 13 to reach the mirror 12. Since it is desirable for the center of the transmission portion 12c included in the mirror 12 to be located on the optical axis of the photographic light source 13, among the light fluxes reflected by the circular light shielding portion 15c, a light flux near the optical axis is transmitted through the transmission portion 12c. In other words, the transmission portion 12c is included in a part of the first reflection portion, and located on the optical axis of the optical system. Since the light flux restriction units 121 and 122 respectively include the openings 121a and 122a near the optical axis of the photographic light source 13, the light flux reflected by the circular light shielding portion 15c enters the light amount detection unit 11 via the openings 121a and 122. In other words, the light amount detection unit detects the amount of light reflected by the second reflection portion and transmitted through the transmission portion. Among light beams emitted toward the subject's eye, light directed to the annular projection portion 15a is transmitted through the annular projection portion 15a to travel toward the subject's eye 28.

Next, light emitted in a direction opposite to the subject's eye will be described. Among light beams emitted in the direction opposite to the subject's eye, light directly entering to the light amount detection unit 11 is restricted for its entry to the light amount detection unit 11 by the light flux restriction unit 121 as described above. As can be understood from FIG. 10, the reflection portions 12a and 12b of the mirror 12 also restrict the entry of the direct light to the light amount detection unit 11. It is obvious that the reflection portions 12a and 12b of the mirror 12 restrict the entry of the diffused reflection light to the light amount detection unit 11.

As illustrated in FIG. 10, the light flux restriction unit 121 restricts entry of the light flux directly entering from the photographic light source 13 to the light amount detection unit 11 to the light amount detection unit 11. The reflection portions 12a and 12b of the mirror 12 and the light flux restriction unit 121 block traveling of the light, which causes diffused reflection light among the light beams emitted in the direction opposite to the subject's eye, to the light amount detection unit 11 side. Further, as illustrated in FIG. 10, the light flux restriction unit 122 blocks traveling of the light, which is transmitted through the opening 121a of the light flux restriction unit 121 among the light causing diffused reflection light, to the light amount detection unit 11 side. In other words, the light flux restriction unit 122 restricts entry of the light flux, which is emitted from the photographic light source 13 and causes diffused reflection light by the inner wall, to the light amount detection unit 11.

As described above, according to the present exemplary embodiment, the entry of the direct light and the diffused reflection light to the light amount detection unit 11 is restricted, while the light flux reflected by the circular light shielding portion 15c, namely the light flux near the optical axis, enters the light amount detection unit 11. Further, the entry of the direct light from the photographic light source 13 and the diffused reflection light from the inner wall to the light amount detection unit 11 is restricted.

Thus, the ophthalmic apparatus according to the second exemplary embodiment can provide the similar effects to those of the first exemplary embodiment. In addition, since the ophthalmic apparatus detects the light reflected by the desired reflection member, the light amount detection unit can accurately detect the amount of emitted light if the amount of light emitted from the light source is large. One of the reasons for realizing this effect is that the amount of light is reduced according to a reflectance.

Further, according to the ophthalmic apparatus of the present exemplary embodiment, since the circular light shielding portion 15c of the ring slit 15 is used as the reflection member, there is no need to provide any new reflection member by using the current configuration, and enlargement of the apparatus can be prevented.

According to the ophthalmic apparatus of the present exemplary embodiment, the reflectance on the reflection surface of the circular light shielding portion 15c is uniform and known. Thus, since the light amount detection unit 11 can detect light with the known and uniform characteristics, the amount of emitted light can be stably measured.

In addition, since the circular light shielding portion 15c of the ring slit 15 is near the annular projection portion 15a through which the light for illuminating the subject's eye 28 passes, the light reflected by the circular light shielding portion 15c is smaller in variance than the light for illuminating the subject's eye 28.

Further, according to the ophthalmic apparatus of the present exemplary embodiment, the entry of the direct light to the light amount detection unit is restricted. Thus, the light amount detection unit can accurately measure the amount of emitted light.

Further, according to the ophthalmic apparatus of the present exemplary embodiment, the entry of the diffused reflection light to the light amount detection unit is restricted. Thus, the light amount detection unit can accurately and stably measure the amount of emitted light.

Furthermore, according to the ophthalmic apparatus of the present exemplary embodiment, since the light amount detection unit can accurately measure the amount of light emitted from the light source, the amount of emitted light can accurately be controlled.

In the present exemplary embodiment, the ophthalmic apparatus includes the light flux restriction unit 121 and the light flux restriction unit 122. However, the present exemplary embodiment is not limited to this configuration. For example, the light flux restriction unit 122 may not be included if diffused reflection light is permitted to enter the light amount detection unit 11.

The light flux restriction unit is not limited to the light flux restriction units 121 and 122. The light flux restriction units of other shapes can be used.

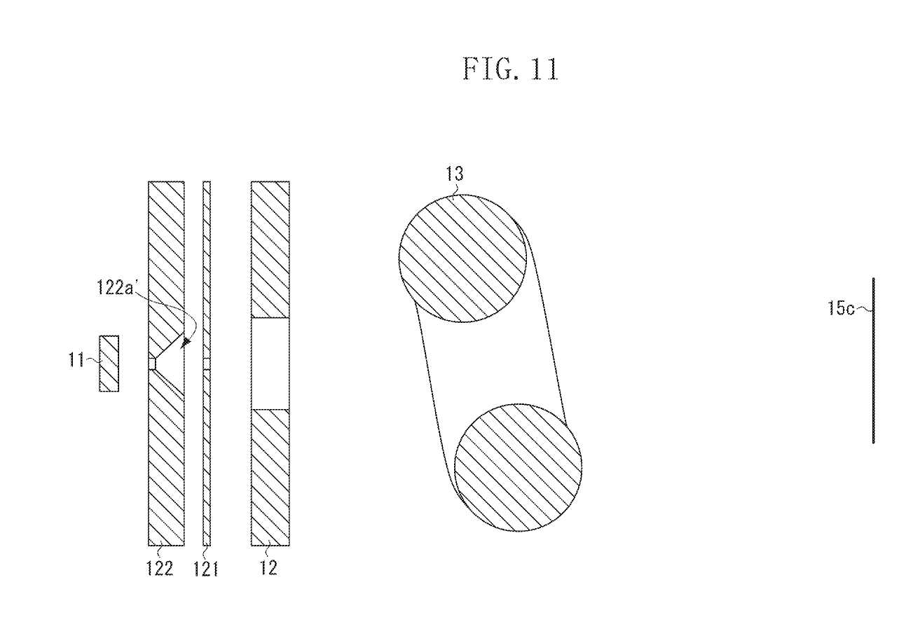

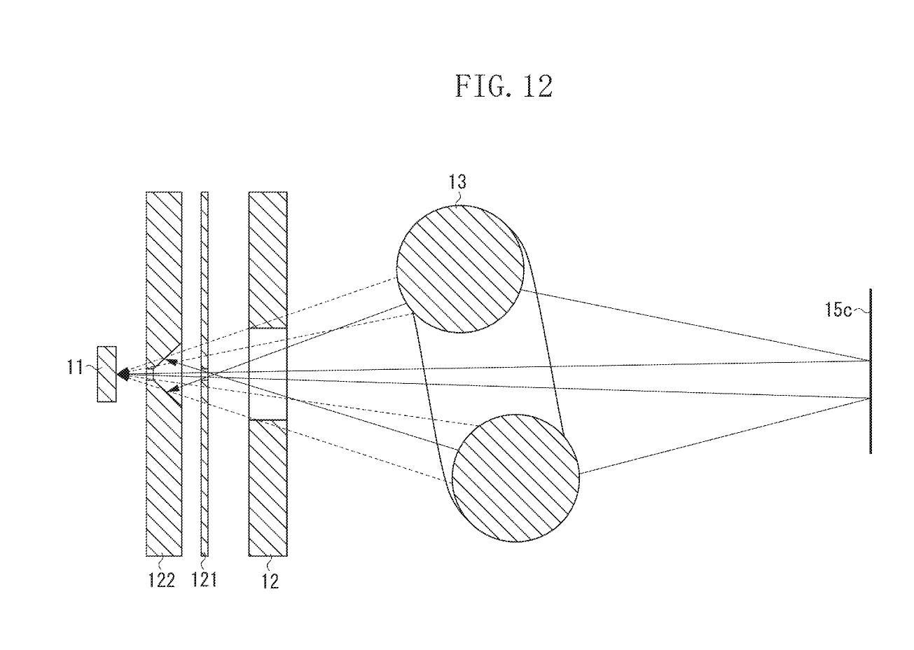

For example, the shape of the opening 122a of the light flux restriction unit 122 illustrated in FIGS. 9 and 10 is not limited to that illustrated in FIGS. 9 and 10. As illustrated in FIGS. 11 and 12, the opening 122a can be formed into a bowl shape. The opening 122a illustrated in FIGS. 11 and 12 is formed to be smaller as approaching the light amount detection unit.

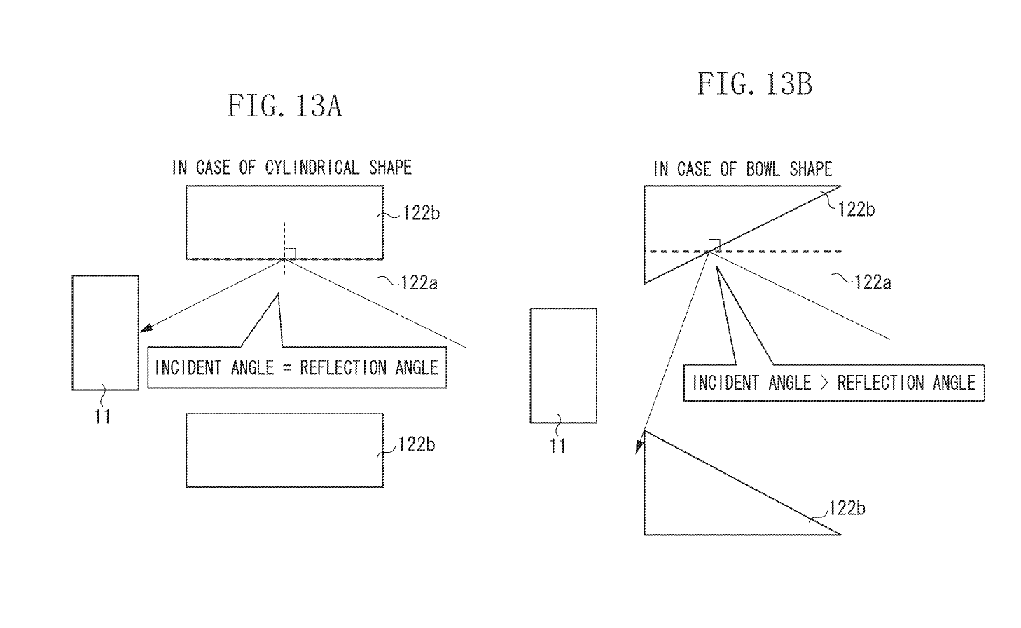

Referring to FIGS. 13A and 13B, effects provided by the bowl shape of the opening 122a will be described. First, as illustrated in FIG. 13A, when the opening 122a has a cylindrical shape, an incident angle and a reflection angle of the light reflected by alight shielding portion 122b are equal to each other. On the other hand, as illustrated in FIG. 13B, when the opening 122a has a bowl shape, the incident angle of the light reflected by the light shielding portion 122b is larger than the reflection angle. The incident angle and the reflection angle illustrated in FIG. 13B are the angles with respect to a portion indicated by a dotted line for comparison with those illustrated in FIG. 13A.

Thus, when the opening 122a has the cylindrical shape, the light that has entered the opening 122a is reflected, for example, once, and reaches the light amount detection unit 11. However, when the opening 122a has the bowl shape, since the reflection angle is smaller than the incident angle, the light is difficult to enter the light amount detection unit 11. As a result, if the light reflected by the bowl-shaped opening 122a reaches the light amount detection unit 11, by repeating reflection, the light is weakened and its influence on the measurement of the amount of emitted light can be reduced.

Next, an ophthalmic apparatus according to a third exemplary embodiment will be described with reference to the drawings.

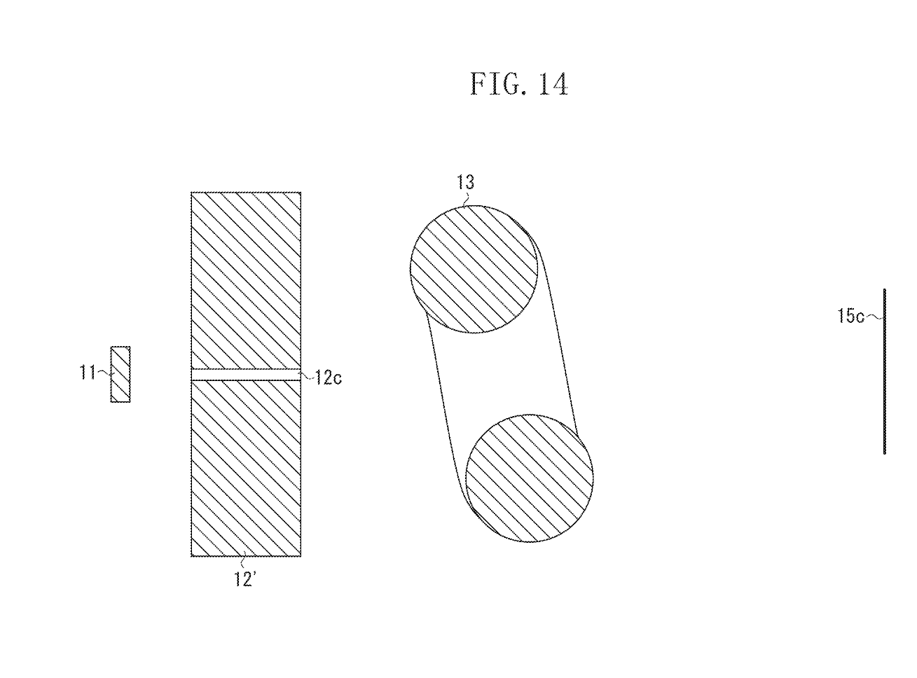

FIG. 14 illustrates a light amount detection unit 11, a mirror 12′, a light source 13, and a circular light shielding portion 15c included in the ophthalmic apparatus according to the third exemplary embodiment.

The ophthalmic apparatus according to the third exemplary embodiment is different from that of the second exemplary embodiment in that neither of light flux restriction units 121 and 122 is included. The ophthalmic apparatus according to the third exemplary embodiment includes the mirror 12′ which has a shape different from that of the second exemplary embodiment.

The mirror 12′ includes a transmission portion 12c which is smaller than that of the mirror 12 of the first exemplary embodiment and having a thickness in an optical axis direction. As in the case of the mirror 12, the mirror 12′ includes reflection portions 12a and 12b and transmission portions 12c, 12d, and 12e. The mirror 12′ does not need to include any of the transmission portions 12d and 12e.

As illustrated in FIG. 14, by forming the transmission portion 12c smaller and longer in the optical axis direction of a photographic light source 13, entry of direct light and diffused reflection light to a light amount detection unit 11 can be restricted.

A size of the transmission portion 12c is designed to prevent direct light from entering the light amount detection unit 11. For example, the size of the transmission portion 12c is determined so as not to include a straight line connecting a light emitting portion of the photographic light source 13 with the light amount detection unit 11. A length of the transmission portion 12c in the optical axis direction is determined so that light directed toward the light amount detection unit 11 side via the transmission portion 12c among light beams emitted from the photographic light source 13 deviates from a path of the transmission portion 12c. This length is determined based on, for example, a distance between the photographic light source 13 and the mirror 12′.

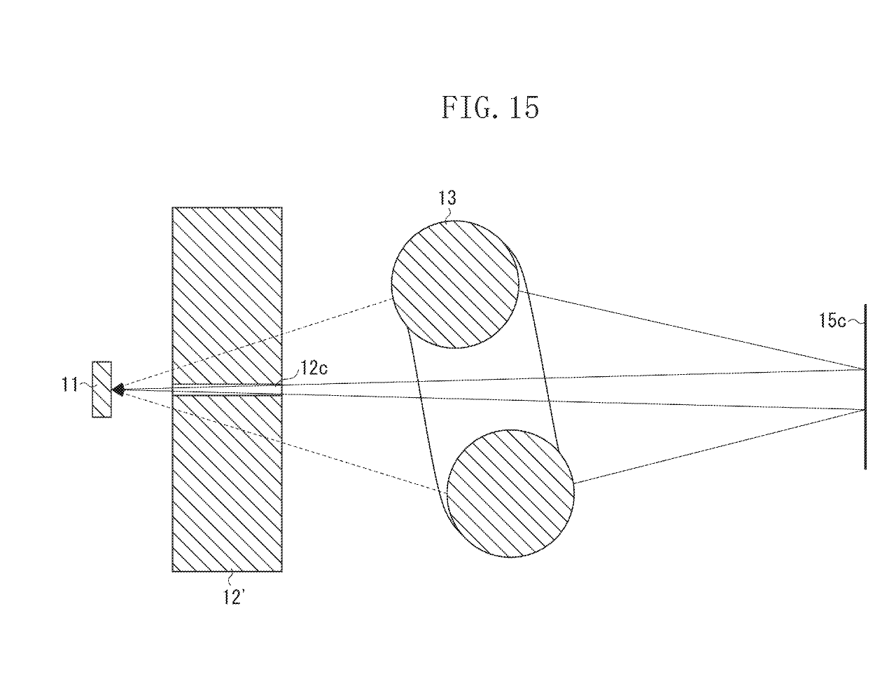

FIG. 15 illustrates a behavior of light emitted from the photographic light source 13. As illustrated in FIG. 15, the transmission portion 12c of the mirror 12′ is smaller than the transmission portion of the mirror 12 of the first exemplary embodiment, and thus light directly entering from the photographic light source 13 to the light amount detection unit 11 cannot pass through the transmission portion 12c. Light causing diffused reflection light and directed from the photographic light source 13 to the light amount detection unit 11 deviates from the path of the transmission portion 12c because the light has an angle with respect to the optical axis if the light enters the transmission portion 12c.

On the other hand, light emitted from the photographic light source 13 toward a subject's eye and reflected by the circular light shielding portion 15c passes through the transmission portion 12c disposed on the optical axis of the photographic light source 13 to enter the light amount detection unit 11.

According to the third exemplary embodiment, the similar effects to those of the second exemplary embodiment can be provided, and the number of members can be reduced.

The above-described present exemplary embodiment includes the mirror 12′ acquired by changing the shape of the mirror 12. However, the exemplary embodiment is not limited to this configuration. Any one of the light flux restriction units 121 and 122 can be formed in a shape similar to the mirror 12′.

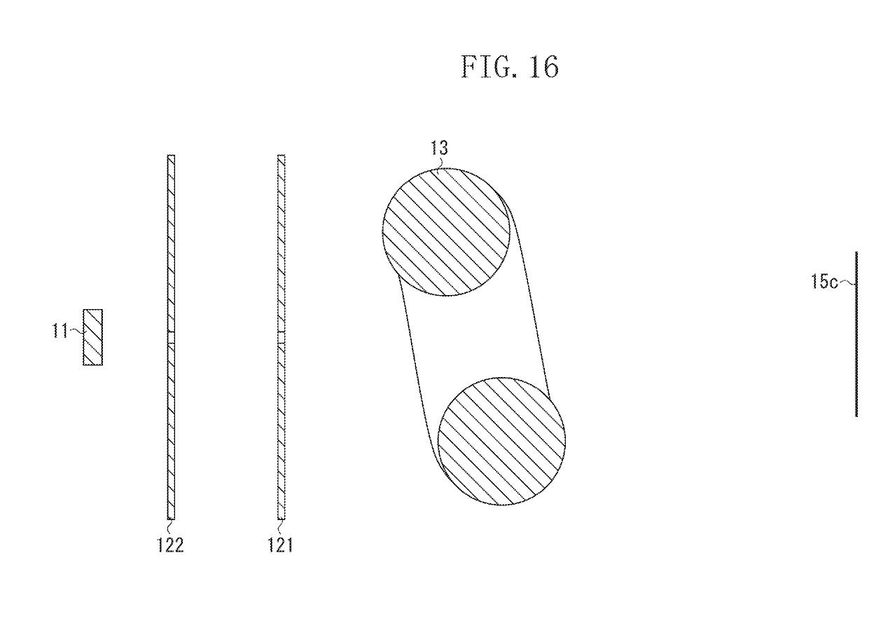

Next, an ophthalmic apparatus according to a fourth exemplary embodiment will be described with reference to the drawings. FIG. 16 is a schematic sectional view illustrating an example of a configuration of a light source unit O1 of the ophthalmic apparatus. FIG. 16 illustrates a light amount detection unit 11, light flux restriction units 121 and 122, a photographic light source 13, and a circular light shielding portion 15c included in the ophthalmic apparatus according to the fourth exemplary embodiment.

Different from the second exemplary embodiment, the ophthalmic apparatus according to the fourth exemplary embodiment does not include a mirror 12.

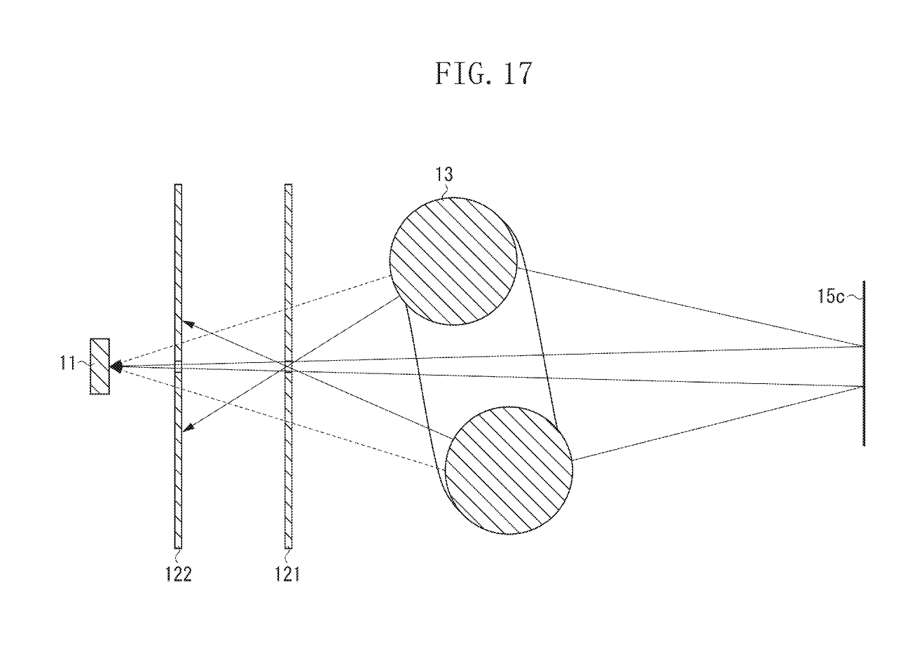

FIG. 17 illustrates a behavior of light emitted from the photographic light source 13. As described above, the light flux restriction unit 121 restricts entry of at least direct light to the light amount detection unit 11. The light flux restriction unit 122 restricts entry of, among light beams emitted from the photographic light source 13 and passed through an opening 121a, at least light causing diffused reflection light to the light amount detection unit 11. On the other hand, light emitted from the photographic light source 13 toward a subject's eye and reflected by the circular light shielding portion 15c passes through a transmission portion 12c disposed on an optical axis of the photographic light source 13 to enter the light amount detection unit 11.

Accordingly, if a mirror 12 is not included, the entry of the diffused reflection light and the direct light to the light amount detection unit 11 is restricted.

Thus, the ophthalmic apparatus according to the fourth exemplary embodiment can provide effects other than those provided by the mirror 12 of the second exemplary embodiment. Further, according to the ophthalmic apparatus of the fourth exemplary embodiment, since no mirror is used, the number of members can be reduced, and the entire apparatus can be downsized.

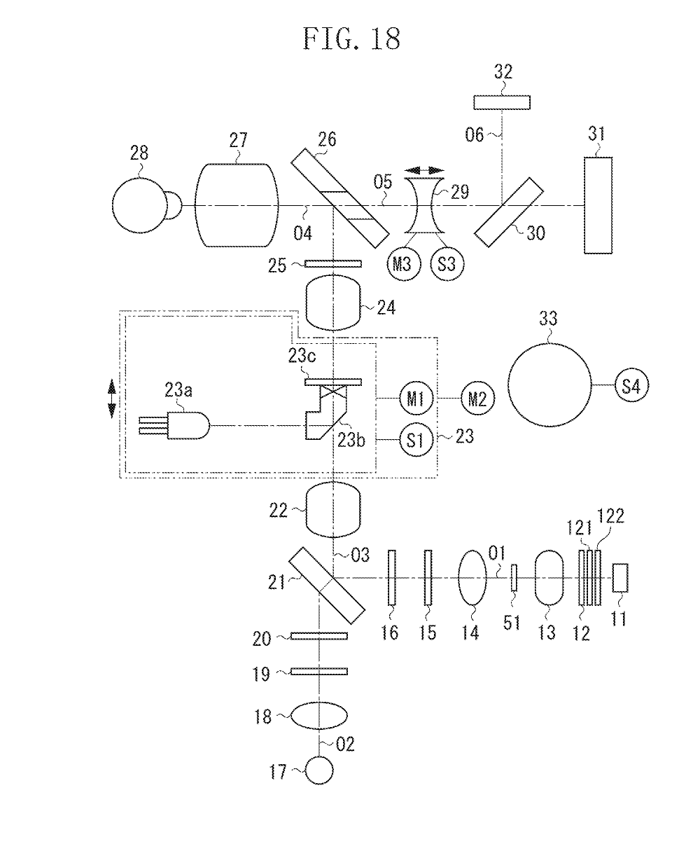

An ophthalmic apparatus according to a fifth exemplary embodiment will be described with reference to the drawings. FIG. 18 schematically illustrates a configuration of the ophthalmic apparatus. Components similar to those described in the above exemplary embodiments are denoted by the same reference numerals, and thus descriptions thereof will be omitted.

The fifth exemplary embodiment is different from the second exemplary embodiment in that the ophthalmic apparatus includes a reflection plate 51. A size of the reflection plate 51 is equal to or nearly equal to, for example, that of a circular light shielding portion 15c included in a ring slit 15, and a reflectance and a surface shape are controlled. For example, a reflectance on a reflection surface of the reflection plate 51 is uniform or nearly uniform. In other words, a reflectance of the reflection plate 51 is known. The reflection plate 51 reflects light emitted from a photographic light source 13 toward a subject's eye in a direction opposite to a direction toward the subject's eye.

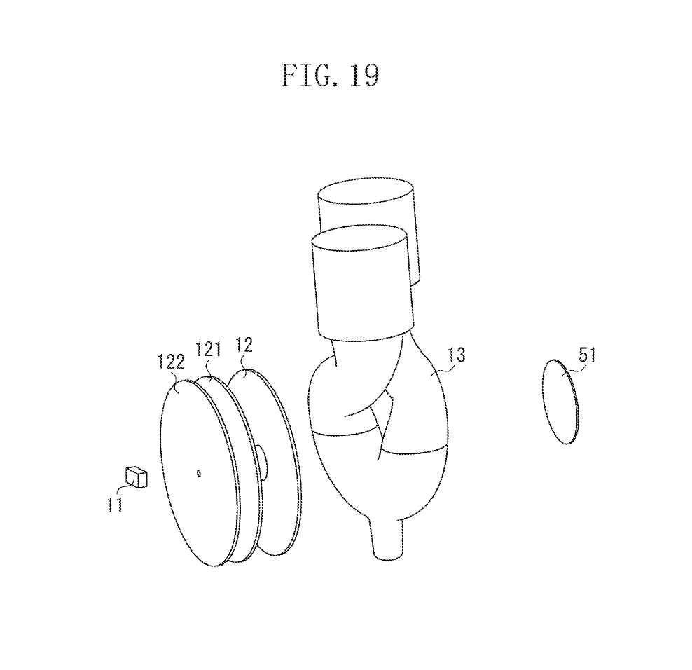

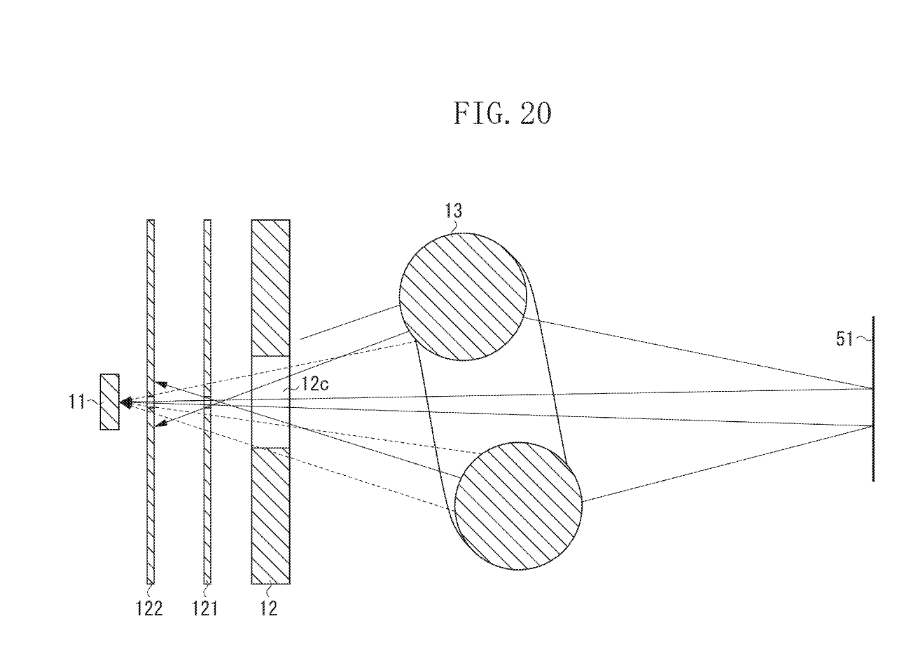

FIG. 19 illustrates a light amount detection unit 11, light flux restriction units 121 and 122, a mirror 12, the photographic light source 13, and the reflection plate 51. FIG. 20 illustrates a cross section of FIG. 19. FIG. 20 also illustrates a behavior of light emitted from the photographic light source 13. As illustrated in FIG. 18, the light flux restriction unit 121 restricts entry of at least direct light to the light amount detection unit 11. The light flux restriction unit 122 restricts entry of, among light beams emitted from the photographic light source 13 and passed through an opening 121a, at least light causing diffused reflection light to the light amount detection unit 11. On the other hand, light emitted from the photographic light source 13 toward a subject's eye and reflected by the reflection plate 51 passes through a transmission portion 12c disposed on an optical axis of the photographic light source 13 to enter the light amount detection unit 11.

Accordingly, if the reflection plate 51 is included in place of the circular light shielding portion 15c, the entry of the diffused reflection light and the direct light to the light amount detection unit 11 can be restricted, while the light reflected by the reflection plate 51 enters the light amount detection unit 11.

Thus, the ophthalmic apparatus according to the fifth exemplary embodiment can provide effects other than those provided by the circular light shielding portion 15c of the second exemplary embodiment. Further, according to the ophthalmic apparatus of the fifth exemplary embodiment, only the refection plate 51 needs to be replaced without replacing a ring slit 15, and thus a member for reflecting light from the photographic light source 13 can easily be replaced.

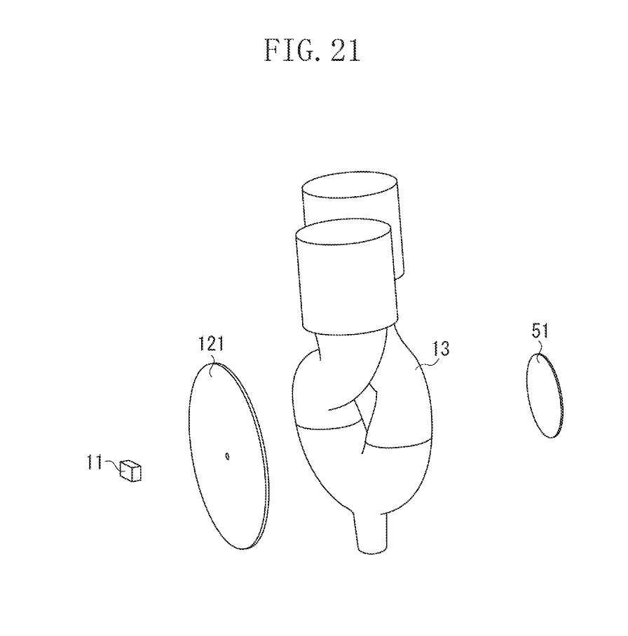

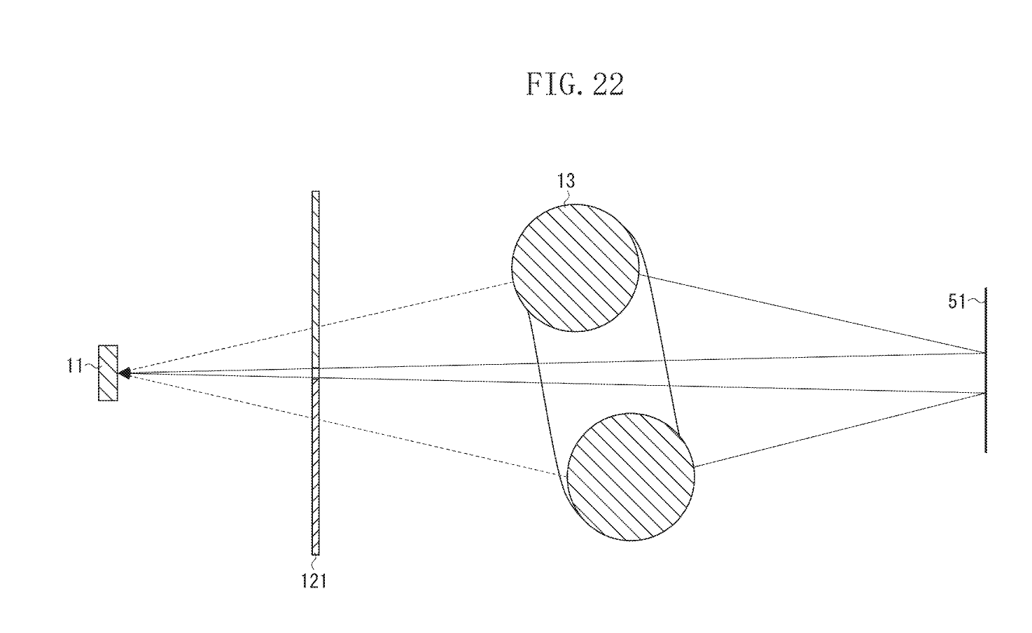

As illustrated in FIG. 21, the present exemplary embodiment need not to include a mirror 12 and a light flux restriction unit 122. In this case, as illustrated in FIG. 22, by disposing the light flux restriction unit 121, at least the entry of the direct light to the light amount detection unit 11 can be restricted. As a result, if the amount of light is large, the amount of light emitted from the photographic light source can be accurately measured.

In the case illustrated in FIGS. 21 and 22, a mirror 12 can be included. More specifically, by including the light flux restriction unit 121 and the mirror 12, the entry of the direct light to the light amount detection unit 11 is restricted, and the mirror 12 can reflect light not directed toward the subject's eye among the light beams generated by the photographic light source 13 toward the subject's eye.

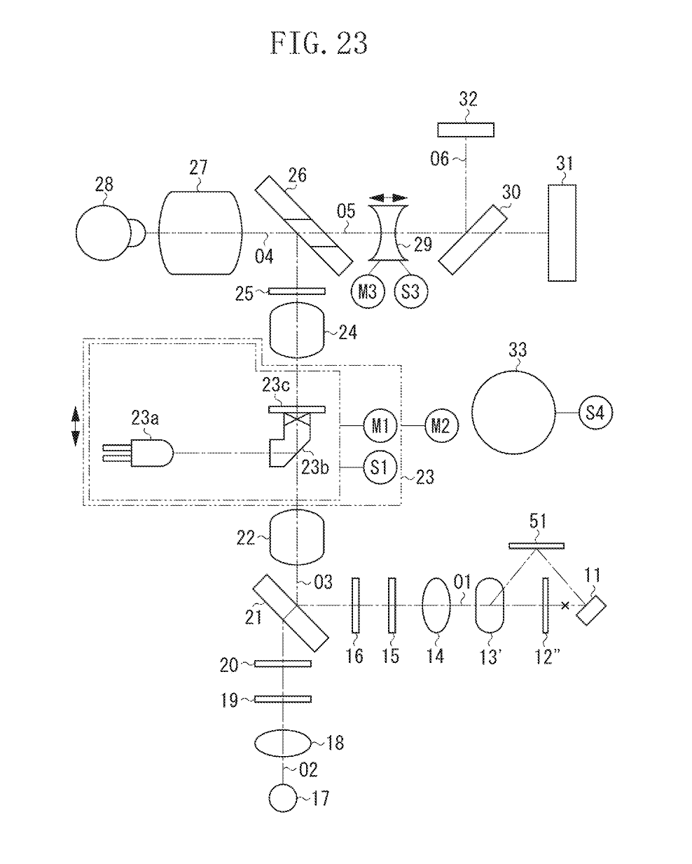

An ophthalmic apparatus according to a sixth exemplary embodiment will be described with reference to the drawings. FIG. 23 schematically illustrates a configuration of the ophthalmic apparatus. Components similar to those described in the above exemplary embodiments are denoted by the same reference numerals, and thus descriptions thereof will be omitted.

The sixth exemplary embodiment is different from the fifth exemplary embodiment in that the ophthalmic apparatus does not include a reflection plate 51 on an optical axis of a photographic light source 13. Further, different from the fifth exemplary embodiment, the ophthalmic apparatus of the sixth exemplary embodiment does not include light flux restriction units 121 and 122. Further, different from the mirror 12 of the fifth exemplary embodiment, the ophthalmic apparatus of the sixth exemplary embodiment includes a mirror 12″ that does not include a transmission portion 12c. Further, the ophthalmic apparatus of the sixth exemplary embodiment includes a rod-shaped photographic light source 13′ different from the photographic light source 13 of the fifth exemplary embodiment. As illustrated in FIG. 23, a light amount detection unit 11 is disposed obliquely to an optical axis of the photographic light source 13′ to receive reflected light from the reflection plate 51. A positional relationship between the light amount detection unit 11 and the reflection plate 51 is determined so that the light amount detection unit 11 can receive the light emitted from the photographic light source 13′ and reflected by the reflection plate 51.

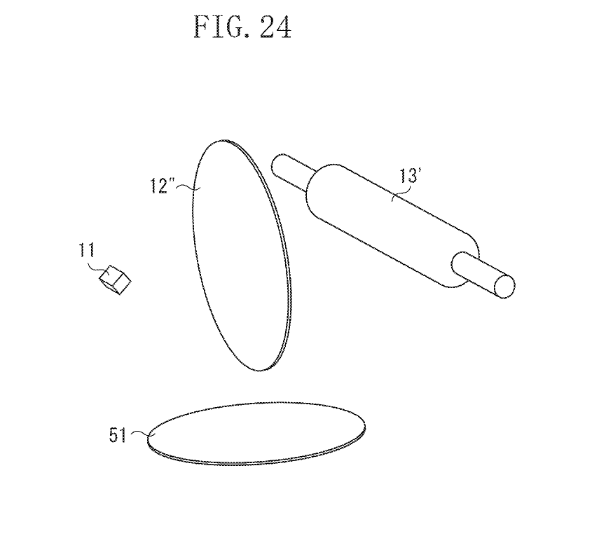

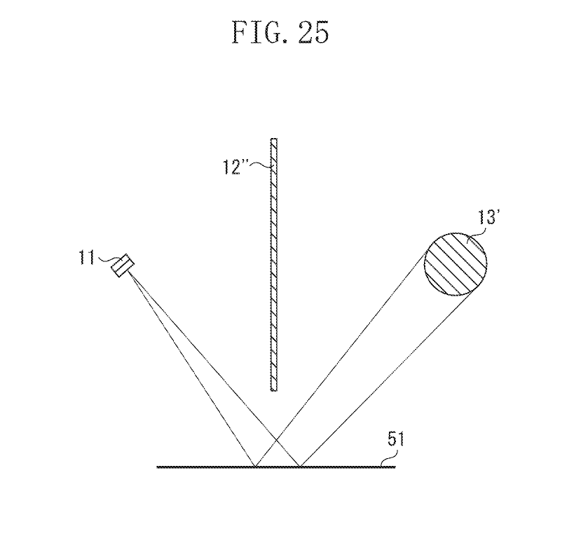

FIG. 24 illustrates the light amount detection unit 11, the mirror 12″, the photographic light source 13′, and the reflection plate 51. FIG. 25 illustrates a cross section of FIG. 24.

As illustrated in FIG. 25, the mirror 12″ restricts entry of, among light beams emitted from the photographic light source 13′, light directly entering to the light amount detection unit 11. In other words, a light flux emitted toward the light amount detection unit 11 is blocked by the mirror 12″ serving as a light flux restriction unit and does not reach the light amount detection unit 11. On the other hand, a part of a light flux emitted from the photographic light source 13′ is reflected by the reflection plate 51 to reach the light amount detection unit 11, and the amount of light is detected. In this case, the reflected light from the reflection plate 51 has known characteristics because a reflectance of the reflection plate 51 is known. Thus, since light amount detection is performed on a light flux with the known characteristics not including direct light from the photographic light source 13′, the light detection can be performed stably. As a result of the detection, stable light control can be realized.

It may not be the mirror 12″ that restricts the entry of the direct light from the photographic light source 13′ to the light amount detection unit 11. For example, a light flux restriction unit 121 having no opening 121a can be used.

In FIG. 23, the reflection plate 51 is located above the mirror 12″. However, the position the reflection plate 51 is not limited to this configuration. For example, in FIG. 23, the reflection plate 51 can be disposed below the mirror 12″.

An ophthalmic apparatus according to a seventh exemplary embodiment will be described with reference to the drawings. Components similar to those described in the above exemplary embodiments are denoted by the same reference numerals, and thus descriptions thereof will be omitted.

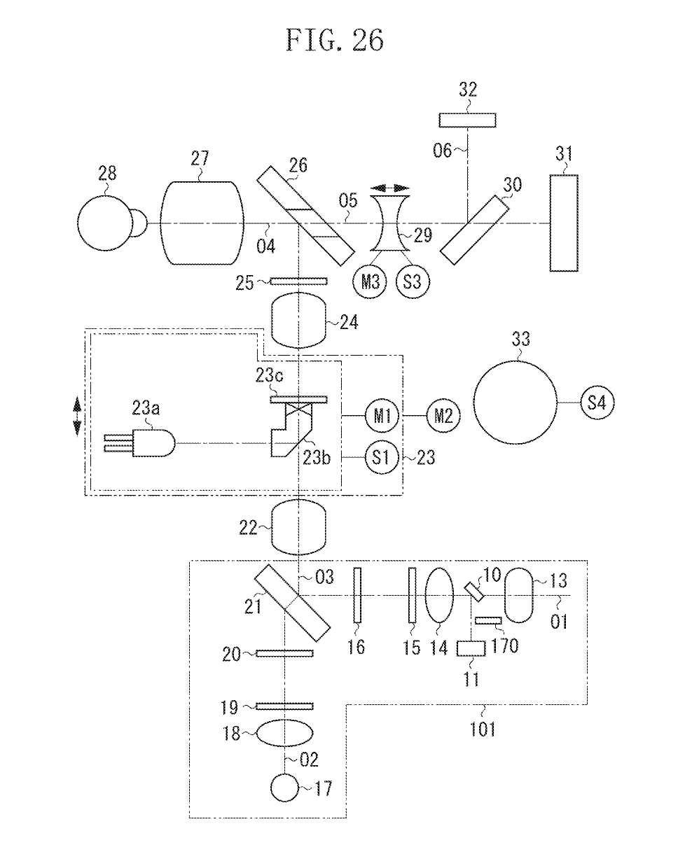

FIG. 26 schematically illustrates a configuration of the ophthalmic apparatus according to the seventh exemplary embodiment. The seventh exemplary embodiment is different from the second exemplary embodiment in that the ophthalmic apparatus includes a mirror 10 in place of the mirror 12. In addition, a position of a light amount detection unit 11 is different from that of the second exemplary embodiment. Further, the seventh exemplary embodiment is different from the second exemplary embodiment in that the ophthalmic apparatus includes a light flux restriction unit 170 without including light flux restriction units 121 and 122.

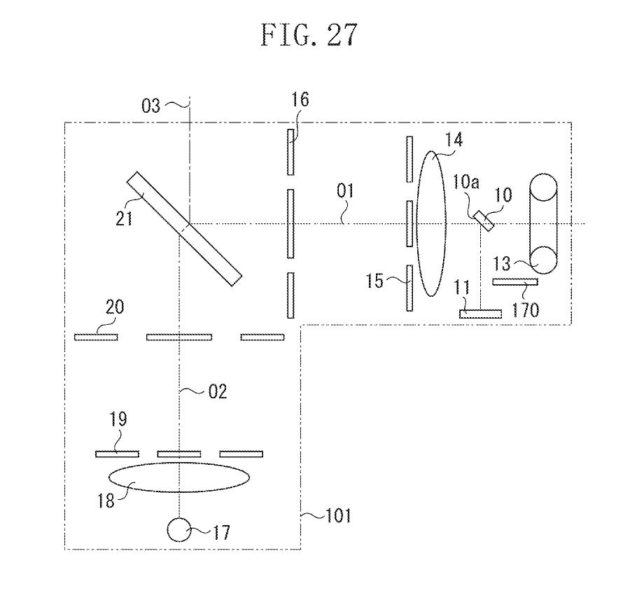

FIG. 27 illustrates a detailed configuration of a photographic light source unit O1 and an observation light source unit O2.

The mirror 10 is, for example, a glass plate disposed between a photographic light source 13 on an optical axis of the photographic light source unit and a photographic ring slit 15, and a surface 10a on a ring slit side of the mirror 10 is formed by aluminum or silver deposition. Alternatively, the mirror 10 can be an aluminum plate. Further, the mirror 10 provides an optical path different from the optical path of the photographic light source unit O1, and the light amount detection unit 11 is located to face the optical path.

The light flux restriction unit 170 is located, for example, on a straight line connecting a light emitting portion of the photographic light source 13 with a light receiving unit 11a of the light amount detection unit 11. The light flux restriction unit 170 restricts entry to the light amount detection unit 11 of light directly traveling from the photographic light source 13 to the light receiving unit 11a of the light amount detection unit 11.

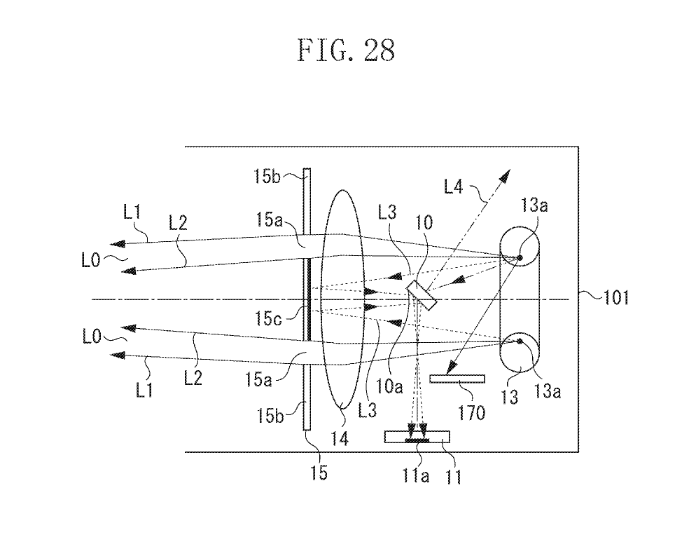

Next, a behavior of light emitted from the photographic light source 13 will be described with reference to FIG. 28.

A light flux L0 is a light flux directed toward a subject's eye among light fluxes radially emitted from an annular light emitting portion 13a of the photographic light source 13. The light flux L0 is an annular light flux that passes through a photographic condenser lens 14 to be condensed toward a fundus of the subject's eye, and then passes through an annular projection portion 15a to form a light flux passes through an anterior segment of the subject's eye 28 into an annular shape. A diameter of the light flux L0 formed into the annular shape is determined based on a size of the annular projection portion 15a. The diameter of the light flux L0 is determined based on light beams L1 and L2. Among the light beams radially emitted from the annular light emitting portion 13a, the light beam L1 passes through the photographic condenser lens 14 from the annular light emitting portion 13a, is restricted by an annular light shielding portion 15b, and then directed toward the fundus of the subject's eye 28. Among the light beams radially emitted from the annular light emitting portion 13a, the light beam L2 passes through the photographic condenser lens 14 from the annular light emitting portion 13a, is restricted by a circular light shielding portion 15c, and then directed toward the fundus of the subject's eye 28.

Among the light beams radially emitted from the annular light emitting portion 13a of the photographic light source 13, a light beam L3 is projected to the light receiving unit 11a included in the light amount detection unit 11. The light beam L3 passes through the photographic condenser lens 14, and is then reflected by the circular light shielding portion 15c of the photographic ring slit 15. The reflected light beam from the circular light shielding portion 15c is returned by a surface 10a of the mirror 10 in a direction where the light amount detection unit 11 is located, and projected to the light receiving unit 11a.

Among the light beams radially emitted from the annular light emitting portion 13a of the photographic light source 13, a light beam L4 is reflected by the mirror 10 to be directed toward an inner wall of a case 101.

The light flux restriction unit 170 located on the straight line connecting the annular light emitting portion 13a of the photographic light source 13 with the light receiving unit 11a restricts entry to the light receiving unit 11a of the light directly traveling from the photographic light source 13 to the light receiving unit 11a.

Thus, the light amount detection unit 11 detects light illustrated as the light beam. L3 among the light beams radially emitted from the annular light emitting portion 13a of the photographic light source 13, and instructs the CPU 61 to stop the light emission when the amount of light reaches the amount of emitted light limited by the CPU 61. Then, the light emission from the photographic light source 13 is stopped via a photographic light source control circuit 62.

As described above, a path is identified for the light beam L3 among the light beams radially emitted from the annular light emitting portion 13a, as the light beam which passes through the photographic condenser lens 14, is reflected by the circular light shielding portion 15c, and then returned to the light receiving unit 11a from the reflection surface 10a. Accordingly, the light amount detection unit 11 actually detects the amount of light based on light located near light for illuminating the subject's eye 28.

Thus, according to the ophthalmic apparatus of the present exemplary embodiment, since the ophthalmic apparatus detects the light reflected by the desired reflection member, the light amount detection unit can accurately detect the amount of emitted light if the amount of light emitted from the light source is large. One of the reasons for realizing this effect is that the amount of light is reduced according to a reflectance.

According to the ophthalmic apparatus of the present exemplary embodiment, since the circular light shielding portion 15c of the ring slit 15 is used as the reflection member, there is no need to provide a new reflection member by using the current configuration, and enlargement of the apparatus can be prevented.

Further, according to the ophthalmic apparatus of the present exemplary embodiment, the reflectance on the reflection surface of the circular light shielding portion 15c is uniform and known. Thus, since the light amount detection unit 11 can detect light with the known and uniform characteristics, the amount of emitted light can be stably measured.

According to the ophthalmic apparatus of the present exemplary embodiment, the light amount detection unit 11 detects the reflected light of the light located near the light passing through the annular projection portion 15a, which is the light for illuminating the subject's eye 28. The amount of light emitted from the light source varies according to the light emitting portion and the light emitting direction. Thus, according to the ophthalmic apparatus of the present exemplary embodiment that actually detects the amount of light based on the light located near the light for illuminating the subject's eye 28, the influence of the variance can be reduced during the detection of the amount of emitted light. In other words, according to the ophthalmic apparatus of the present exemplary embodiment, the amount of emitted light can accurately be detected.

Since the circular light shielding portion 15c of the ring slit 15 is near the annular projection portion 15a through which the light for illuminating the subject's eye 28 passes, the light reflected by the circular light shielding portion 15c is smaller in variance with respect to the light for illuminating the subject's eye 28.

Further, according to the ophthalmic apparatus of the present exemplary embodiment, the entry of the direct light to the light amount detection unit is restricted. Thus, the light amount detection unit can accurately measure the amount of emitted light.

Further, according to the ophthalmic apparatus of the present exemplary embodiment, the entry of the diffused reflection light to the light amount detection unit is restricted. Thus, the light amount detection unit can accurately and stably measure the amount of emitted light.

Furthermore, according to the ophthalmic apparatus of the present exemplary embodiment, since the light amount detection unit can accurately measure the amount of light emitted from the light source, the amount of emitted light can accurately be controlled.

In addition, since the light source is optimized to illuminate the subject's eye 28, light near the optical axis of the light source is more stable as compared to light not near the optical axis. Thus, according to the ophthalmic apparatus of the present exemplary embodiment, since the light amount detection unit 11 measures the reflected light near the optical axis of the light source, the amount of emitted light can accurately be detected.

The positions of the mirror 10 and the light amount detection unit 11 are not limited to those of the present exemplary embodiment. Various changes can be made as long as the light amount detection unit 11 can receive the light reflected by the circular light shielding portion 15c.

An ophthalmic apparatus according to an eighth exemplary embodiment will be described with reference to the drawings. Components similar to those described in the above exemplary embodiments are denoted by the same reference numerals, and thus descriptions thereof will be omitted.

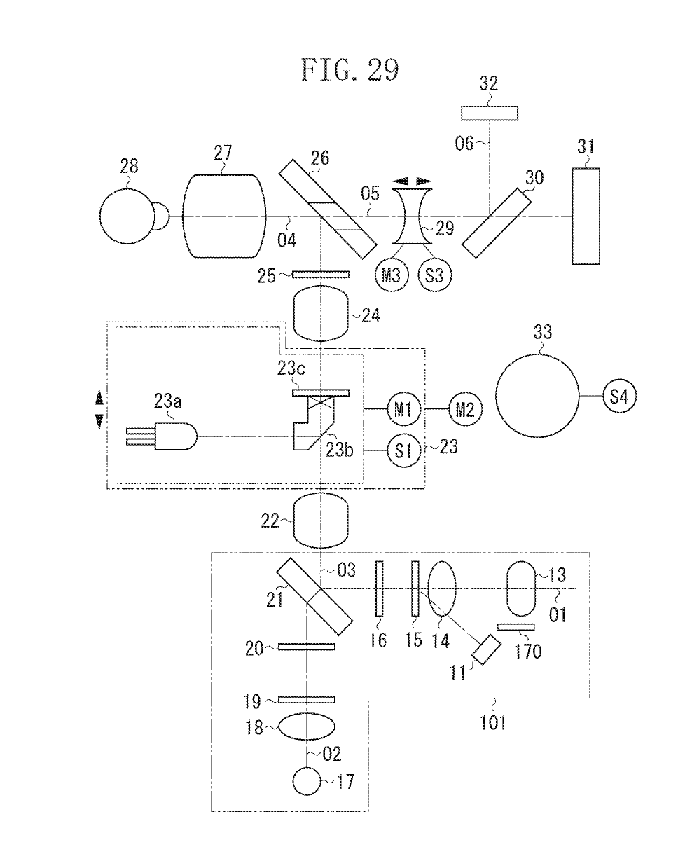

FIG. 29 schematically illustrates a configuration of the ophthalmic apparatus according to the eighth exemplary embodiment. The eighth exemplary embodiment is different from the second exemplary embodiment in that the ophthalmic apparatus does not include a mirror 12. In addition, positions of a light amount detection unit 11 and a light flux restriction unit 170 are different from those of the sixth exemplary embodiment.

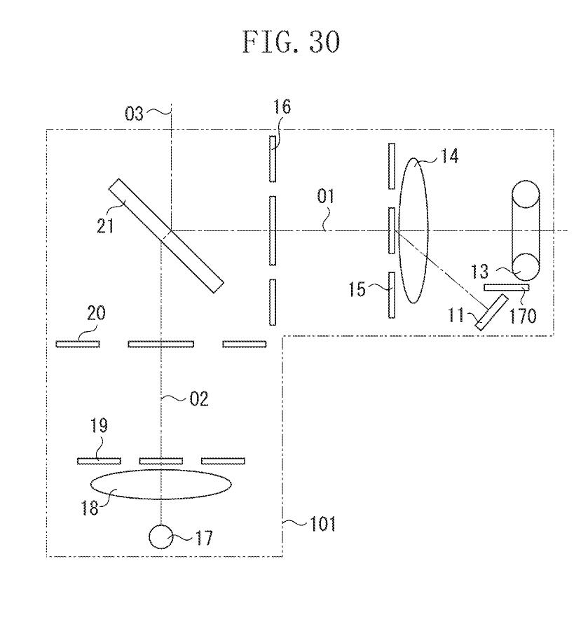

FIG. 30 illustrates a detailed configuration of a photographic light source unit O1 and an observation light source unit O2.

The light amount detection unit 11 has its light receiving surface set obliquely to a ring slit 15 side. The light flux restriction unit 170 is located, for example, on a straight line connecting a light emitting portion of a photographic light source 13 with a light receiving unit 11a of the light amount detection unit 11. The light flux restriction unit 170 restricts entry to the light amount detection unit 11 of light directly traveling from the photographic light source 13 to the light receiving unit 11a of the light amount detection unit 11.

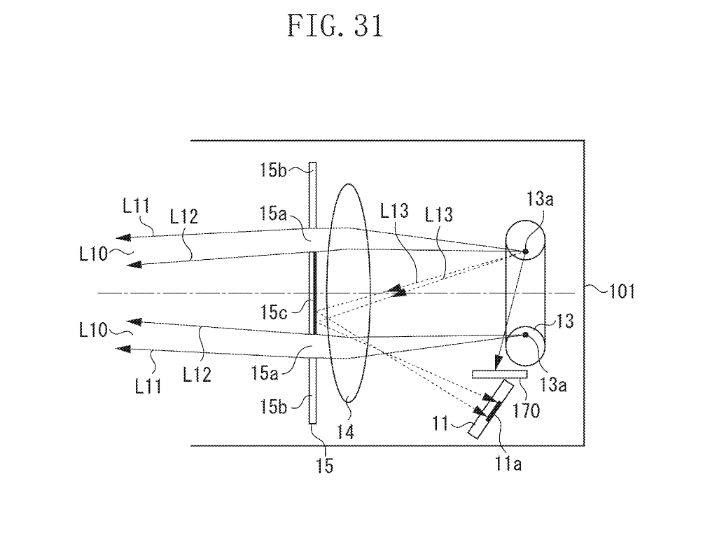

Next, referring to FIG. 31, a behavior of light emitted from the photographic light source 13 will be described.

A light flux L10 is, among light fluxes radially emitted from an annular light emitting portion 13a of the photographic light source 13, a light flux directed toward a subject's eye. The light flux L10 is an annular light flux that passes through a photographic condenser lens 14 to be condensed toward a fundus of the subject's eye, and then passes through an annular projection portion 15a to form a light flux passes through an anterior segment of the subject's eye 28 into an annular shape. A diameter of the light flux L10 formed into the annular shape is determined based on a size of the annular projection portion 15a. The diameter of the light flux L10 is determined based on light beams L11 and L12. Among the light beams radially emitted from the annular light emitting portion 13a, the light beam L11 passes through the photographic condenser lens 14 from the annular light emitting portion 13a, is restricted by an annular light shielding portion 15b, and then directed toward the fundus of the subject's eye 28. Among the light beams radially emitted from the annular light emitting portion 13a, the light beam L12 passes through the photographic condenser lens 14 from the annular light emitting portion 13a, is restricted by a circular light shielding portion 15c, and then directed toward the fundus of the subject's eye 28.

Among the light beams radially emitted from the annular light emitting portion 13a of the photographic light source 13, a light beam L13 is projected to the light receiving unit 11a included in the light amount detection unit 11. The light beam L13 passes through the photographic condenser lens 14, and is then reflected by the circular light shielding portion 15c of the photographic ring slit 15. The reflected light beam from the circular light shielding portion 15c is projected to the light receiving unit 11a.

The light flux restriction unit 170 located on the straight line connecting the annular light emitting portion 13a of the photographic light source 13 with the light receiving unit 11a restricts entry to the light receiving unit 11a of the light directly traveling from the photographic light source 13 to the light receiving unit 11a.

Thus, the light amount detection unit 11 detects light illustrated as the light beam L13 among the light beams radially emitted from the annular light emitting portion 13a of the photographic light source 13, and instructs the CPU 61 to stop the light emission when the amount of light reaches the amount of emitted light limited by the CPU 61. Then, the light emission from the photographic light source 13 is stopped via a photographic light source control circuit 62.

As described above, a path is identified for the light beam L13 among the light beams radially emitted from the annular light emitting portion 13a, as the light beam which passes through the photographic ring slit 15, is reflected by the circular light shielding portion 15c, and then returned to the light receiving unit 11a. Accordingly, the light amount detection unit 11 actually detects the amount of light based on light located near light for illuminating the subject's eye 28.

Thus, the ophthalmic apparatus according to the present exemplary embodiment can provide the similar effects to those of the sixth exemplary embodiment.

The position of the light amount detection unit 11 is not limited to that of the present exemplary embodiment. Various changes can be made as long as the light amount detection unit 11 can receive the light reflected by the circular light shielding portion 15c.

In the above-described exemplary embodiments, the light flux restriction units 170, 121, and 122 are included. However, the present invention is not limited to this arrangement. For example, no light flux restriction unit can be disposed by setting the light amount detection unit 11 in a position where no or little direct light enters from the photographic light source 13. Concerning the position where no or little direct light enters from the photographic light source 13, for example, the light amount detection unit 11 can be located directly below the photographic light source 13, and the light receiving surface of the light receiving unit 11a can be located in a direction orthogonal to the optical axis of the photographic light source 13. Further, concerning the position where no or little direct light enters from the photographic light source 13, for example, the light amount detection unit 11 can be located on the optical axis of the photographic light source 13, and the light receiving surface of the light receiving unit 11a can be located in a direction orthogonal to the optical axis of the photographic light source 13. In other words, in the above-described exemplary embodiments, the light flux restriction unit is not essential.

Further, in the above-described exemplary embodiments, the amount of light emitted from the photographic light source 13 is detected. However, the present invention is not limited to this arrangement. For example, not the amount of light emitted from the photographic light source 13 but the amount of light emitted from the observation light source 17 can be measured. According to the above-described exemplary embodiments, the fundus camera is described as the ophthalmic apparatus. However, the present invention is not limited to the fundus camera, and can be applied to other ophthalmic measuring apparatuses.

Aspects of the present invention can also be realized by a computer of a system or apparatus (or devices such as a CPU or an MPU) that reads out and executes a program recorded on a memory device to perform the functions of the above-described embodiments, and by a method, the steps of which are performed by a computer of a system or apparatus by, for example, reading out and executing a program recorded on a memory device to perform the functions of the above-described embodiments. For this purpose, the program is provided to the computer for example via a network or from a recording medium of various types serving as the memory device (e.g., computer-readable medium).

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures, and functions.

This application claims priority from Japanese Patent Applications No. 2011-167053 filed Jul. 29, 2011, No. 2011-167054 filed Jul. 29, 2011, and No. 2011-167055 filed Jul. 29, 2011, which are hereby incorporated by reference herein in their entirety.

What is claimed is:

1. An ophthalmic apparatus comprising:
an optical system configured to illuminate a subject's eye with light generated by a light source;
a first reflection portion including a reflection surface for reflecting the light generated by the light source and a transmission portion; and
a light amount detection unit configured to detect an amount of light generated by the light source via the transmission portion,
wherein the first reflection portion is disposed in a direction opposite to a direction of the light generated by the light source toward the subject's eye.

2. The ophthalmic apparatus according to claim 1, wherein the reflection surface is disposed in the direction of the light generated by the light source toward the subject's eye.

3. The ophthalmic apparatus according to claim 2, wherein the light amount detection unit is disposed in the direction opposite to the direction of the light generated by the light source toward the subject's eye, and
the first reflection portion is disposed between the light amount detection unit and the light source.

4. The ophthalmic apparatus according to claim 1, wherein the transmission portion is disposed on an optical axis of the optical system.

5. The ophthalmic apparatus according to claim 4, wherein the light amount detection unit is disposed on the optical axis of the optical system.

6. The ophthalmic apparatus according to claim 1, further comprising a ring slit disposed in a position nearly conjugate with an anterior segment of the subject's eye and including a second reflection portion configured to reflect a part of the light generated by the light source on an optical axis of the optical system,
wherein the light amount detection unit detects an amount of light reflected by the second reflection portion and transmitted through the transmission portion.

7. The ophthalmic apparatus according to claim 6, further comprising a first restriction unit configured to restrict entry of light, which directly travels from the light source to the light amount detection unit, into the light amount detection unit.

8. The ophthalmic apparatus according to claim 7, wherein the first restriction unit is disposed on a straight line connecting a light emitting portion of the light source with the light amount detection unit.

9. The ophthalmic apparatus according to claim 8, wherein the first restriction unit includes a first opening through which light can pass, and
the first opening is disposed on the optical axis.

10. The ophthalmic apparatus according to claim 9, further comprising a second restriction unit configured to restrict entry of at least light other than light reflected by the second reflection portion among the light passed through the first opening, into the light amount detection unit, and including a second opening through which the light can pass on the optical axis.

11. The ophthalmic apparatus according to claim 1, wherein the transmission portion is radially disposed toward an outer circumference of the first reflection portion.

12. The ophthalmic apparatus according to claim 1, further comprising a light emission control unit configured to control light emission of the light source according to an amount of light detected by the light amount detection unit.

13. The ophthalmic apparatus according to claim 1, wherein the light source includes a xenon tube.

14. The ophthalmic apparatus according to claim 1, wherein the light source includes a light-emitting diode (LED).

15. The ophthalmic apparatus according to claim 1, wherein the first reflection portion includes a planar mirror.
